(19) United States
(12) Patent Application Publication
(10) Pub. No.: US 2022/0328198 A1
(43) Pub. Date: Oct. 13, 2022

(54) EFFICIENTLY TRAINING AND EVALUATING PATIENT TREATMENT PREDICTION MODELS

(71) Applicant: University of Pittsburgh - of the Commonwealth System of Higher Education, Pittsburgh, PA (US)

(72) Inventors: Ajay D. Wasan, Pittsburgh, PA (US); Andrea G. Gillman, Pittsburgh, PA (US)

(21) Appl. No.: 17/716,279

(22) Filed: Apr. 8, 2022

Related U.S. Application Data
(60) Provisional application No. 63/173,072, filed on Apr. 9, 2021.

(51) Int. Cl. G16H 50/50; G06N 20/00

(52) U.S. Cl. CPC G16H 50/50; G06N 20/00

(57) ABSTRACT

Systems and methods are described for training a treatment prediction model using patient profiles. The training can include applying a machine-learning technique that causes the treatment prediction model to learn to predict a likelihood that a given patient will respond to a particular medical treatment according to a one or more criteria. A first subset of predictive features are identified from an expanded set of predictive features that are most predictive of whether a given patient will respond to the particular medical treatment according to the one or more criteria. The treatment prediction model is configured to generate predictions from values for the first subset of predictive features that are most predictive, without requiring values for a second subset of predictive features that are less predictive than features from the first subset. The treatment prediction model can be applied to generate a treatment response prediction for a new patient.

### CROSS-REFERENCE TO RELATED APPLICATIONS

This application claims the benefit of priority to U.S. Provisional Application Ser. No. 63/173,072, filed on Apr. 9, 2021. The disclosure of the prior application is considered part of and is incorporated by reference in its entirety into the disclosure of the present application.

### BACKGROUND

### 1. Technical Field

This specification relates to predictive models, including methods, systems, and apparatus for training and evaluating machine-learning models configured to predict patient responses to one or more modalities of medical treatment.

### 2. Background Discussion

Complaints of pain are the most common reason patients seek healthcare in the United States, and the second most common reason that patients attend primary care visits.

As part of a Learning Health Systems approach, the National Academy of Medicine and the National Institutes of Health (NIH) have stressed the importance of collecting patient-reported outcomes (PROs) in pain treatment to accurately track patients' progress and reduce clinician bias in treatment evaluations. As there is currently no validated biological measure of a patient's reported pain level, self-reports are considered the “gold standard” for pain outcomes and are widely accepted as primary and secondary endpoints in FDA-approved trials. When collected consistently during pain treatment, high-quality PROs can be used to determine treatment effects using scientifically valid observational study designs.

However, there is currently little understanding of which treatments for chronic pain are most likely to be effective based on a patient's presenting characteristics (e.g., the patient's “phenotype”). Moreover, developing predictive models that meet the demands of clinical applications can be technically challenging due to the disparate locations of patient data, presence of patient identifying information, incomplete patient data and gaps in PRO surveys, changes in available data sources, frequent addition of patient records that are unaccounted for in static models, and the computational expense required to train and run the models.

### SUMMARY

This specification describes technologies for training and evaluating patient treatment prediction models to predict the likelihood of a patient realizing a clinically meaningful improvement in a medical/health condition responsive to a specified treatment modality. A patient treatment prediction model can be a machine-learning model configured to process as inputs values of features from a personalized patient treatment prediction profile that describes a range of presenting characteristics (e.g., a “phenotype”) of a patient. For example, patient-specific values of features related to demographic, mental and physical health, and pain characteristics of a patient can be processed to assess the probability of a patient responding to a particular treatment or combination of treatments (e.g., medications, injections, therapies, etc.). Moreover, through use of multiple, independently trained predictive models that each correspond to a different treatment modality, treatment response predictions can be obtained for multiple candidate treatment modalities. A clinician and patient can assess the absolute and/or relative likelihoods of the patient responding to the different treatments, and the predictions can, at least in part, guide the clinician's and/or patient's selection of particular treatment(s) for a condition exhibited by the patient (e.g., chronic pain).

Traditionally, predictive models related to patient care have encountered a range of technical challenges. To start, it can be difficult to obtain all of the data necessary to train an effective model as the data must be collected from disparate sources with different formatting, structure, and access requirements. The quantity and quality of patient data available for training the models also can be insufficient to achieve adequate model performance. For example, the data often includes gaps where no data was submitted, errors where incorrect information was submitted, and identifying data that cannot be released from a secure system due to patient privacy regulations. Moreover, the large number of features required by some predictive models can unduly increase the number of parameters and overall size of the models—which not only slows processing and increases computational expense but can also deter use of the models by clinicians and patients due to the burden or inconvenience of collecting all of the inputs necessary for the models to run. Furthermore, as additional patient data and new features become available, systems may lack efficient methods of incorporating the new data into previously trained models. Implementations of the subject matter described in this specification can, in certain cases, address one or more of these challenges.

In general, one innovative aspect of the subject matter described in this specification can be embodied in methods that include the actions of obtaining, by a system comprising one or more computers, patient profiles for a plurality of patients, wherein each patient profile corresponds to a particular patient and includes (i) values for an expanded set of predictive features about the particular patient and (ii) a target response classification that indicates whether the particular patient responded to a particular medical treatment according to one or more criteria; training, by the system, a treatment prediction model using the patient profiles, including applying a machine-learning technique that causes the treatment prediction model to learn to predict, based on predictive features from a patient profile for a given patient, a likelihood that the given patient will respond to the particular medical treatment according to the one or more criteria; identifying, by the system, a first subset of predictive features from the expanded set of predictive features that are most predictive of whether a given patient will respond to the particular medical treatment according to the one or more criteria; configuring the treatment prediction model to generate predictions from values for the first subset of predictive features that are most predictive, without requiring values for a second subset of predictive features that are less predictive than features from the first subset; and applying the treatment prediction model to generate a treatment response prediction for a new patient. Other embodiments of this aspect include corresponding computer systems, apparatus, and computer programs recorded on one or more computer storage devices, each configured to perform the actions of the methods.

In general, another innovative aspect of the subject matter described in this specification can be embodied in methods that include the actions of obtaining, by a system comprising one or more computers, patient data that describes information about a patient and a medical condition of the patient; generating, by the system and based on the patient data, a patient profile for the patient, wherein the patient profile comprises values for a plurality of predictive features and the plurality of predictive features include one or more shared predictive features that are each processed by two or more treatment prediction models of a plurality of treatment prediction models, wherein the plurality of treatment prediction models each corresponds to a different medical treatment modality of a plurality of medical treatment modalities; generating treatment response predictions for the patient for each of the plurality of medical treatment modalities, including for each medical treatment modality: processing, with the treatment prediction model that corresponds to the medical treatment modality, at least a subset of the plurality of predictive features from the patient profile to generate a treatment response prediction for the medical treatment modality, wherein the two or more treatment prediction models each process the one or more shared predictive features to generate the treatment response predictions for the corresponding medical treatment modalities; and outputting information about the treatment response predictions for the patient for one or more of the plurality of medical treatment modalities. Other embodiments of this aspect include corresponding computer systems, apparatus, and computer programs recorded on one or more computer storage devices, each configured to perform the actions of the methods.

The foregoing and other embodiments can each optionally include one or more of the following features, alone or in combination. In particular, one embodiment includes all the following features in combination.

The subject matter described in this specification can be implemented in particular embodiments so as to realize one or more of the following advantages. First, the treatment prediction models and associated systems can provide a shared decision-making tool to help patients and healthcare providers select personalized treatment regimens that are most likely to benefit the patient, while minimizing healthcare costs that might otherwise be incurred by prescribing treatments that are less likely to be effective to a particular patient. Second, by pulling patient data from multiple sources into a single database or into multiple databases within a single system, data from each of the sources can be correlated, and re-formatted/re-structured so as to provide a more complete picture of a patient and provides additional features that can be assessed for inclusion in the predictive models. Third, by anonymizing the patient data through actions such as filtering and removal of identifying information and encryption of patient identifiers (e.g., patient medical record numbers), the anonymized patient data can be transferred to untrusted or less trusted systems that from an original secure/trusted environment where the non-anonymized patient data is initially stored (e.g., a hospital or health care provider's system). The system to which the patient data is transferred can more efficiently train and execute the predictive models, thereby freeing resources of the original secure/trusted computing environment. Fourth, by periodically or regularly importing data from external sources into a centralized system or database more frequently than the model is trained or re-trained, updated patient data can be collected in smaller increments that avoids overly large data transfers that risks consuming undue bandwidth and processing resources of the systems at the endpoints of the transfer. Efficiency in the importing process can further be realized by importing new data or changed data into the treatment profiles database rather than re-writing the entire database during each update. Fifth, additional patient records can be directed to training the models by imputing values of features that are missing in the originally sourced data. Sixth, the size of the predictive model can be reduced while retaining high performance by configuring the model to generate predictions on a reduced, core set of predictive features. By reducing the number of predictive features that are required or most strongly advised to provide as input to a model, the burden of using the model may be lowered thereby promoting higher usage rates by clinicians and patients alike (e.g., since patients may be required to report less information about their demographics, pain characteristics, outcomes, and the like). Additionally, smaller models are typically advantageous since they can be run faster and can require fewer computational resources. Seventh, by periodically re-training the model on updated patient treatment profiles, the set of core predictive features can be dynamically re-assessed from time to time. Thus, as more patient records and/or additional sources become available, the mix of core predictive features can be improved by adding features that were not previously recognized in the core set and/or discarding previously-identified core features that are subsequently determined to be less predictive. In this way, model performance can improve over time without unduly increasing the size of the model. Eighth, shared predictive features can be identified across multiple models so that multiple models can be efficiently evaluated without requiring collection or computation of entirely different sets of inputs for each model.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example environment for training and evaluating patient treatment prediction models.

FIG. 2A is a flowchart of an example process for importing data into a patient treatment profiles database.

FIG. 2B is a flowchart of an example process for generating a patient treatment prediction profile.

FIG. 3 is a flowchart of an example process for training and validating a patient treatment prediction model.

FIG. 4 is a flowchart of an example process for updating a patient treatment prediction model.

FIG. 5 is a flowchart of an example process for evaluating patient treatment prediction models to generate treatment response predictions.

FIG. 6A is a plot of a receiver operating characteristic (ROC) curve showing the true positive rate vs. the false positive rate of the random forest models developed in the example study described in this specification. The area under the ROC curve (AUROC) was 0.65 for this model.

FIG. 6B is a plot of a calibration curve showing the observed averages versus predicted values of the random forest models developed in the example study described in this specification. Calibration error was calculated to be 0.04 for this treatment model.

FIG. 7 is a block diagram of an example computing system.

Like reference numbers and designations in the various drawings indicate like elements.

### DETAILED DESCRIPTION

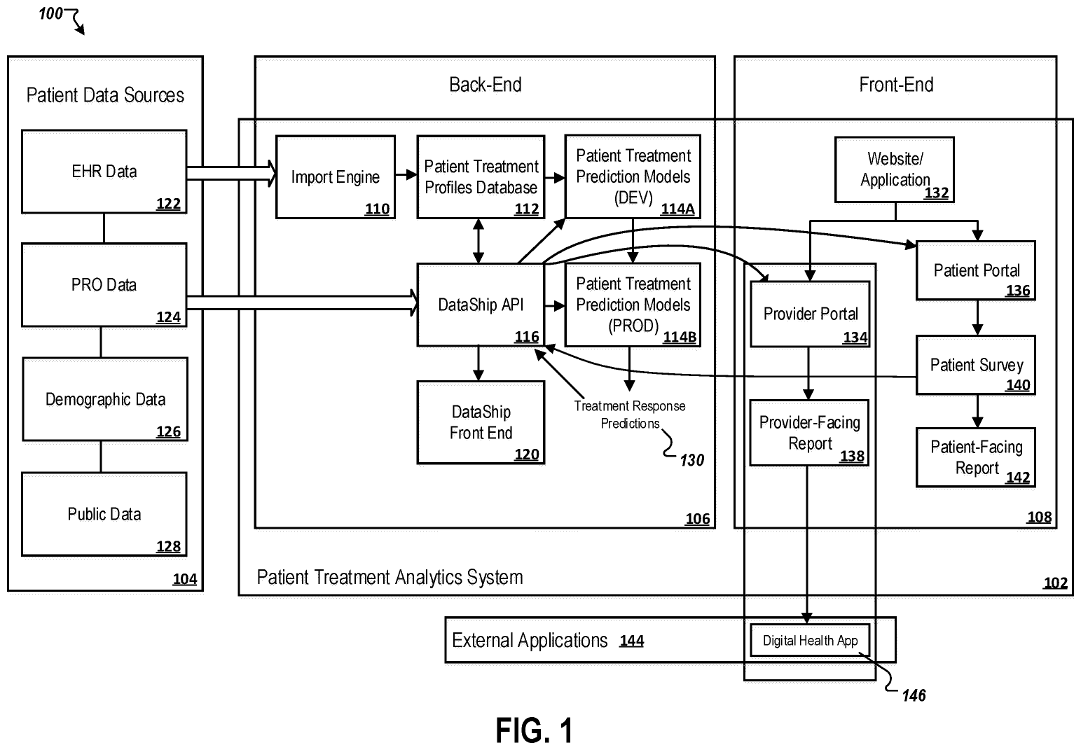

FIG. 1 is a block diagram of an example computer environment 100. The environment 100 includes a patient treatment analytics system 102, patient data sources 104, and external applications 144. In general, the systems shown in environment 100 can be implemented on one or more computers in one or more locations. Preferably, the backend components 106 of analytics system 102 are physically or logically distinct from patient data sources 104 and front-end components 108. The components in environment 100 can be communicably coupled over one or more networks, e.g., local area networks (LANs), wireless area networks (WANs), the Internet, or other networks.

In general, analytics system 102 is configured to train, evaluate, and report outputs of one or more patient treatment prediction models 114. The patient treatment prediction models 114 are machine-learning models that process predictive inputs to generate treatment response predictions 130. Each treatment prediction model 114 is independently trained to generate a treatment response prediction 130 indicating a likelihood that a patient will respond to a different treatment modality. In other words, each treatment prediction model 114 generates predictions for different treatment modalities from other ones of the prediction models 114. A treatment modality represents a particular treatment or combination of treatments. In the field of pain management, for example, models 114 can be trained to predict responses to treatments such as naproxen medication, schedule III/IV opioid medications, anticonvulsant medications, muscle relaxant medications, meloxicam medication, aspirin medication, celecoxib medication, antidepressant medications, cervical epidural injections, interlaminar lumbar epidural injections, muscle and tendon injections, abdominal and pelvic blocks, pain pumps and other implanted devices, behavioral medicine, integrative medicine, rehabilitation therapies (e.g., occupational therapy, physical therapy), orthotics and assistive devices, or combinations of two or more these. In the example study described in this specification, a random forest model (comprising a set of decision trees) were shown to effectively implement a treatment prediction model 114. However, other types of machine-learning models are also suitable such as artificial neural networks, regression models, support-vector machines, and naive Bayes models. The treatment response prediction 130 can be provided in any suitable form, including a binary classification (e.g., a value indicating whether the patient likely will or will not respond to a particular treatment modality) or a probability score indicating a probability or likelihood that the patient will respond to a particular treatment modality.

Training a treatment prediction model 114 requires training data, which is generally obtained or derived from sourced patient data initially stored in one or more patient data sources 104. The patient data sources 104 include electronic healthcare record (EHR) data 122 (also referred to as electronic medical record (EMR) data), patient-reported outcome data 124, patient demographic data 126, public data 128, or combinations of these. Each of these patient data sources 104 may be stored jointly or separately across one or more databases and under the control of one or more custodians (who may or may not be related). For example, EHR data 122 may be stored in a first healthcare provider's system, PRO data 124 may be stored in the first healthcare provider's system, a second healthcare provider's system, or submitted directly from a patient's computer, demographic data 126 may be stored in the same or different private systems, and public data 128 may be publicly available through other servers (e.g., via the Internet). In some implementations, each of the patient data sources 104 are maintained separately, thereby requiring separate collection by the analytics system 102. In other implementations, all or some of the patient data sources 104 may be combined within one or more databases under control of a single entity. For example, a healthcare provider may store EHR data 122, but patients may submit PRO data 124, demographic data 126, or both, directly to the healthcare provider for incorporation into EHR data 122. EHR data 122, PRO data 124, and demographic data 126 typically contain private or sensitive patient information, and can be encrypted and stored in secure environments to protect the data from leaks to unauthorized parties.

EHR data 122 stores electronic healthcare/medical records for individual patients. Examples of information stored in EHR data 122 for a given patient includes the patient's diagnoses for one or more medical conditions over a period of time (e.g., last day, week, month, 6 months, 1 year, 2 years, 5 years, 10 years, 15 years, 20 years), chronic pain diagnoses (e.g., back neck or spine pain, lower back pain, neuropathic pain or nerve injuries, fibromyalgia, migraine, osteoarthritis, rheumatoid arthritis, other arthritis or arthropathy, other musculoskeletal pain, other chronic pain conditions), co-morbidities (e.g., anxiety, congestive heart failure, connective tissue disorder, depression, diabetes, hypothyroidism, irritable bowel syndrome, PTSD, seizure disorders, sleep apnea, thyroid disorders), prescriptions or concurrent medication use (e.g., schedule II/III/IV opioids, antidepressants, NSAIDs, muscle relaxants), tobacco, alcohol, or illicit drug use, or combinations of these. In some implementations, diagnoses are labeled according to their International Classification of Diseases (ICD) codes.

PRO data 124 stores patient-reported information, such as responses provided by patients in at-home or in-clinic surveys. Examples of information stored in PRO data 124 include self-reports of physical and/or mental states related to a medical condition (e.g., chronic pain) at baseline and after a period of time (e.g., 60+/−30 days) indicative of an outcome from the recommendation of one or more treatment modalities by a physician. For example, a patient with lower back pain may be prescribed physical therapy for a period of two months to help alleviate the back pain. The patient can record information describing the intensity of the back pain and the impact on physical function as a result of the back pain at baseline (e.g., at or shortly before the start of physical therapy) and after a specified period of time undergoing physical therapy (e.g., 60+/−30 days).

The patient can also report information about his or her overall impression of change in the medical condition (e.g., lower back pain) at the end of the reporting period. As described in this specification, the outcome measures from PRO data 124 can be applied to determine target classifications as to whether the patient successfully responded to a particular treatment modality (e.g., physical therapy) according to one or more criteria. In some implementations, PRO data 124 includes additional patient-reported information such as education level (e.g., highest education level completed), whether the patient is involved in legal action related to the medical condition (e.g., pain), work status (e.g., whether the patient is currently employed), disability assistance status (e.g., whether the patient receives one or more forms of government subsidies/assistance), marital status, co-habitation status, duration of pain experience (e.g., how long the patient has experienced the medical condition), body map information (e.g., information identifying areas on a body map where the patient identified pain, or a number of regions selected on the pain body map), information indicating whether the patient experienced the medical condition (e.g., chronic pain) as a child, neuropathic pain score, body map cluster membership, information indicating the presence of pain in major anatomical regions (e.g., abdomen, ankle, arm, buttocks, chest, elbow, foot, hand, head, hip knee, leg, lower back, neck, pelvis/groin, shoulder, upper back, wrist), information that contributes to a pain behavior score, information that contributes to a pain interference score, information that contributes to mental health measures (e.g., PTSD score, opioid misuse index, HEAL positive outlook t-score, history of psychiatric hospitalization, PROMIS anxiety t-score, PROMIS depression t-score, PROMIS global mental health T-score), information that contributes to physical function measures (e.g., PROMIS global physical health t-score, PROMIS sleep disturbance t-score).

Demographic data 126 includes patient characteristics describing demographics of the patient such as age, sex, race, and address or zip code of residence. In some implementations, demographic data 126 is already provided in EHR data 122 or is self-reported and stored in PRO data 124. In general, there may be overlap in information among the various patient data sources 104, and some information in this example as belonging to one of the EHR data 122, PRO data 124, or demographic data 126 can alternatively be provided in another one of the data sources 104. Public data sources 128 can be accessed to obtain additional information about circumstances of the patient. For example, the patient's ZIP code may be used to access information about the median income, crime, and/or poverty levels of the county or area of residence of the patient.

The backend 106 of analytics system 102 includes an import engine 110 configured to access all or some of the patient data sources 104 and import relevant information from these sources 104 into the patient treatment profiles database 112. Import engine 110 presents authentication credentials to access patient data sources 104. In some implementations, the import engine 110 is granted limited access to patient data sources 104 that allows the import engine 110 to obtain a limited portion of data stored in the data sources 104 (e.g., only data that is authorized and relevant to functions of the treatment prediction models 114). Importation events can be triggered by user input, or can be automated to occur periodically or otherwise on a predefined schedule (e.g., daily, weekly, or monthly). Before or during the importation process, the systems can anonymize patient data to protect patient's private and identifying information. For example, information such as residence addresses and names can be stripped from the imported data (or not transferred in the first instance). Additionally, patient identifiers such as patient-specific EHR/EMR medical record numbers can be encrypted or otherwise obfuscated to prevent individual patient records from being related back to the identities of the patients. In some implementations, import engine 110 runs queries on the patient data sources 104 that filters and limits the amount of information imported during each importation session. Large amounts of data may be stored in patient data sources 104, and it would be costly in terms of bandwidth-consumption, importation time, and computational expense to import the entire set of relevant data during each session. Since much of the patient data will have been previously imported, import engine 110 can cooperate with the patient data sources 104 to identify and import only new or changed patient data that was not previously imported in a prior session. Importation can also be time-limited, for example, by importing data from patient records entered only in a most recent period of time (e.g., 1 week, 1 month, 3 months, 6 months, 1 year, 5 years).

Beyond merely pulling and replicating sourced patient data from patient data sources 104, import engine 110 also organizes and joins data from disparate sources, computes values of custom parameters that are derived from (but not explicitly defined in) the sourced patient data, and records values of various predictive features from the patient data in patient treatment profiles database 112. A patient treatment profile comprises a collection of predictive (or potentially predictive) features for an individual patient. For example, a patient treatment profile can include dozens of features extracted or derived from the patients' EHR/EMR data 122, PRO data 124, demographic data 126, and/or public data 128. In some implementations, the values of the features defined by a patient treatment profile are stored in a standardized structure or format that are suitable for processing by the patient treatment prediction models 114. In some implementations, import engine 110 derives values for all or some of the following features: Charlson Comorbidity Index, PTSD score (e.g., calculated by summing the number of PTSD checklist questions selected by a patient in a PRO survey), pain experience duration, opioid score (e.g., calculated by summing the number of opioid misuse checklist items selected by the patient in the PRO survey), PainDETECT neuropathic pain score (e.g., calculated based on patient responses to nine questions related to identifying neuropathic components in patients with back pain), zip code conversion features (e.g., converts the patient's zip code to state of residence, county of residence, and other variables indicating socioeconomic status), dynamic ICD-10 category variables (e.g., calculated from ICD-10 diagnosis codes in the patient's EMR/EHR data by removing all but the first 3 alphanumeric characters (e.g., M15, M79, etc.) to find categories that are present in at least 5% of the current patient population), diagnosis category flag variables (e.g., flags if a patient has a diagnosis in a designated lookup table), medication category flag (e.g., checks medication orders and flags if patient has a medication prescription in a designated look-up table; medication category flags are used to (1) flag patients who received a specific treatment for inclusion in predictive analytics training datasets and (2) to flag patients who were prescribed a specific medication concurrently with another treatment), procedure category flags (e.g., checks procedure orders and flags if patient has a procedure order in a designated look-up table; procedure category flags are used (1) to flag patients who received a specific treatment for inclusion in predictive analytics training datasets and (2) to flag patients who were given a specific treatment concurrently with another treatment), body map region features (e.g., checks spaces selected on a pain body map and flags if patient has selected a space in a designated body region, e.g. head, neck, foot, etc), and body map cluster membership. In some implementations, patient treatment profiles database 112 is apportioned into multiple sections that each store patient profiles corresponding to a different treatment modality. Each section of the database 112 corresponding to a particular treatment modality can be applied to training the respective prediction model 113 related to that treatment modality.

The predictive features are not limited to those described above. In some implementations, sourced patient data can include additional information that enables extraction or determination of additional predictive features such as body-mass index (BMI), pain behavior t-score, insurance information (e.g., whether the patient has private medical insurance, government-sponsored medical insurance, auto insurance, and/or other types or categories of insurance), disability status, race, gender, laboratory testing results on biological samples from the patient, and genetic or epigenetic information from the patient.

Patient treatment profiles for training prediction models 114 also store target prediction response values. The target prediction response value indicates whether a patient sufficiently responded to a particular treatment modality with respect to improvement of a particular medical condition (e.g., chronic pain) over a period of time following administration of the treatment modality. For example, the target prediction response value can be a binary value indicating that the patient either did or did not sufficiently respond to a particular treatment modality. In the example pain treatment study described in this specification, a successful response required satisfaction of at least one of the following thresholds at three month's follow-up after baseline: (1) ≥30% improvement in average pain intensity on a 0-10 numeric scale over the last 7 days, (2) ≥5 points improvement in the PROMIS Physical Function T-Score, and/or (3) a report of “Very Much Improved” (the highest patient-rated category of improvement) on the overall Impression of Change scale. The improvement in average pain intensity can be calculated based as—(follow-up pain intensity−baseline pain intensity)/baseline pain intensity. The exception was the Behavioral Medicine dataset, where the responder outcome analyzed was a combined response benchmark of minimal clinically important differences of either (1) ≥20% improvement in average pain intensity, (2) ≥2 points improvement in physical function, and/or (3) a report of “Slightly Improved” or “Very Much Improved” on the overall Impression of Change scale. In other implementations, different criteria or thresholds can be applied to fit the particular application and treatments at issue.

Import engine 110 is also configured to identify gaps in patient data imported from the patient data sources 104 and, where necessary or appropriate, to impute values of predictive features in the patient profiles that could not be directly extracted or derived from the patient data sources 104 as a result of the identified gaps. In some implementations, imputing a value of a continuous-variable feature involves assigning an average value of the feature from other patient profiles as the value for the feature where a data gap was identified. Imputing a value of a continuous variable-feature can involve assigning a null value of the feature that specifically reflects the identified data gap. In practice, null values can themselves be predictive, and can provide predictive value despite the data gap.

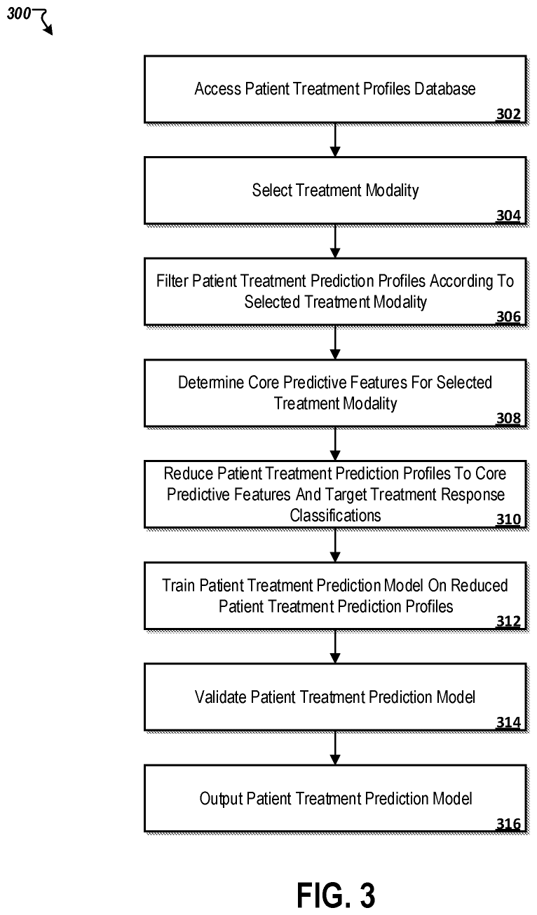

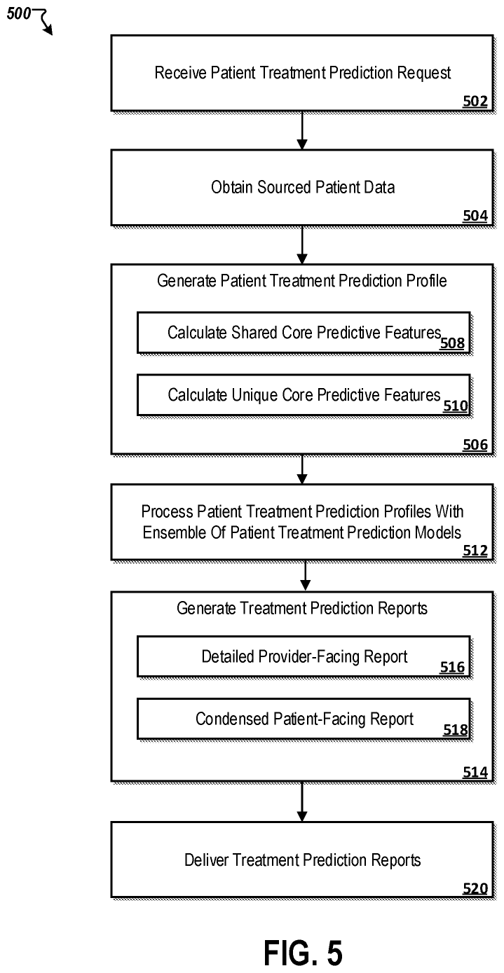

Analytics system 102 processes records of patient treatment profiles from database 112 using a machine-learning algorithm to train patient treatment prediction models 114A. Additional detail on the training process is described with respect to FIG. 3. When a training phase is completed, treatment models 114A are placed into production as treatment models 114B. The production treatment models 114 can receive patient profiles for new patients (e.g., patients who were not represented in the profiles used for training), and processes the patient profiles to generate a treatment response prediction 130. Further detail on the process for evaluating treatment prediction models 114 to generate treatment response predictions 130 is described with respect to FIG. 5.

Analytics system 102 can also implement an application programming interface (API) to receive patient data from one or more patient data sources. For example, as shown in FIG. 1, patients, providers, or both can use the DataShip API 116 to push patient data (e.g., EHR/EMR data 122, PRO data 124, demographic data 126 and/or public data 128) to the analytics system 102. The pushed data generally includes all information about a patient that the system 102 needs to generate a sufficiently complete patient treatment profile (including all information necessary to generate values for core predictive features) that can be processed by one, some, or all of the patient treatment prediction models 114 to produce one or more treatment response predictions 130. The analytics system 102 can also store information received or calculated through the DataShip API within patient treatment profiles database 112. The system 102 can also implement a front end 120 for the API 116, allowing users to submit patent data through the front end 120.

The front-end portion 108 of analytics system 102 further allows patients and providers to interface with the back-end 106, e.g., to submit requests for new treatment response predictions, and to obtain reports describing the predictions. Patients and providers can access the system 102 through a website or web application 132 rendered in a browser or a native application 132 installed on a user device. The website/application 132 can include separate portals 134, 136 for providers and patients, respectively. In the patient portal 136, patients can input data from which predictive features are extracted or derived, including demographic information, descriptions of the medical condition (e.g., pain descriptions), and feedback used to assess the outcome of a treatment modality. For example, the patient-provided data can be submitted in a survey 140. Survey data 140 can be received by the back-end system 106 via an API (not shown), import engine 110 generates feature values for a patient profile, and the applicable predictive features are processed by the suite of patient prediction models 114B to generate treatment response predictions 130 for one or more treatment modalities. Reports 138 and 142 can be published in the provider and patient portals, respectively (or otherwise delivered to the patient by email, postal mail, or other distribution means). In some implementations, the provider-facing report 138 provides more detail than the patient-facing report 142. For example, the provider-facing report 138 may provide a detailed breakout of the individual treatment response predictions 130 for each treatment modality modeled (e.g., including each medication, injection, therapy, etc.). Patients may not require or comprehend the same level of detail, and therefore can be provided a “summary” report that focuses on classes of treatments or a subset of treatment modalities of interest. Analytics system 102 can also provide reports of treatment response predictions 130 to external applications 133, such as digital health application 146 (e.g., on a FITBIT or smartphone tracker).

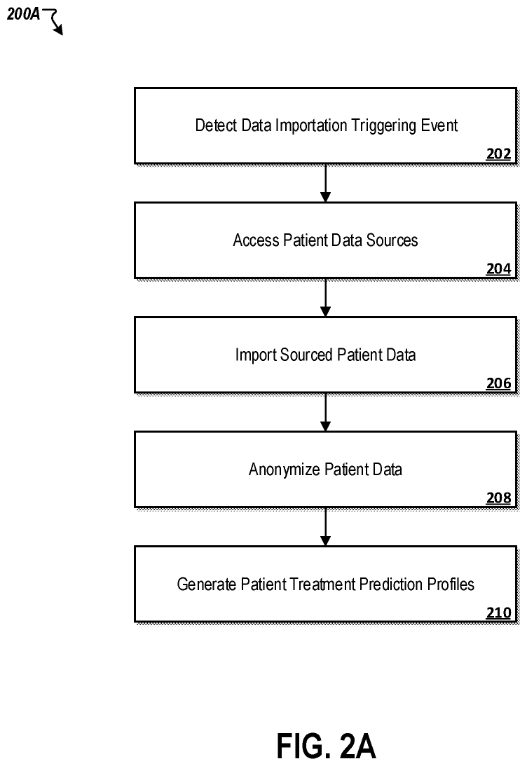

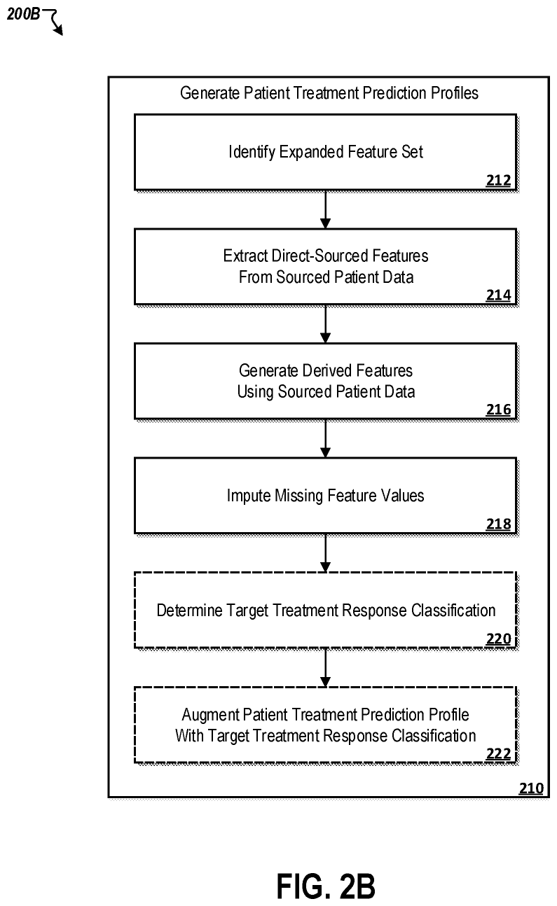

FIG. 2A is a flowchart of an example process 200A for importing data into a patient treatment profiles database. In some implementations, process 200A is carried out by an import engine, e.g., import engine 110. The import engine detects an importation triggering event (202). The triggering event can be based on user input requesting that importation initiate immediately, or can be based on a timer or schedule that causes the import engine to execute on a regular basis (e.g., nightly). The import engine accesses patient data sources (204), such as EMR/EHR data, demographic data, PRO data, and the like. The import engine imports the sourced patient data (206) and prepares the data for structured recording in a patient treatment profile database. Since the patient data is brought outside the provider's system, the data can be anonymized to protect patient identity (208). Further, information relating to the patient's identity such as unique EMR/EHR numbers can be encrypted. Import engine generates patient treatment prediction profiles, and stores the profiles in the database. Where PRO data is available, the import engine computes a target treatment response classification for the patient and records the target classification in the database for use in training a prediction model.

FIG. 2B is a flowchart of an example process 200B for generating a patient treatment prediction profile. Process 200B expands upon operations involved in generating a patient treatment prediction profile. The system (e.g., import engine 110 or other components of analytic system 102) identifies an expanded feature set of the patient treatment prediction profile. The expanded feature set is a relatively large feature space that encompasses all features hypothesized as potentially predictive of whether a patient will successfully respond to one or more treatment modalities. For example, the expanded feature set may encompass all or most of the patient characteristics that can be extracted or derived from the original sourced patient data, on the assumption that any of these characteristics or features is potentially predictive of a patient's likelihood of responding to a given treatment. The system extracts values for direct-sourced features from the sourced patient data (214). Direct-sourced features are features (e.g., date of birth, sex) that can be directly extracted from the sourced patient data without derivation of custom variables. Values of derived features are then calculated from the sourced patient data (216), and the system imputes values for features where a data gap was identified (218). When a patient reports outcomes from a treatment modality after an observational period, the system can then determine the target treatment classification for the patient according to one or more criteria (220), and the patient treatment prediction profile is augmented with the target classification (along with values of the expanded feature set) (222).

FIG. 3 is a flowchart of an example process 300 for training and validating a patient treatment prediction model. The process 300 can be performed with a training subsystem of patient treatment analytics system, e.g., system 102. To start, the system accesses the patient treatment profiles database 302 where many patient treatment prediction profiles are stored (302). The treatment prediction model will be trained to predict treatment responses for a specific treatment modality (and different models can be trained for different treatment modalities). Accordingly, the system selects the desired treatment modality (304) and filters the profiles in the patient treatment profile database to include those profiles corresponding to the selected treatment modality (306). Other profiles corresponding to other treatment modalities are excluded from the training data set.

In some implementations, the system determines a “core” set of predictive features for the selected treatment modality (308). The core predictive features are generally a subset of the expanded set of features that are determined to be most predictive of patients' treatment responses. For example, the expanded feature set may contain dozens of potentially predictive features, but some of these features may, in practice, be non-predictive or may exhibit very low predictive power relative to other features. Therefore, relatively few (e.g., 10-15) features can be selected for inclusion in the core set of features. The core predictive features can be determined according to any suitable statistical method. In some cases, the core features can be determined before training the predictive model. In other cases, the predictive model is first trained on all or most of the predictive inputs in the expanded set, and the model is then analyzed to identify the most predictive features. In either case, the model is configured to generate patient treatment predictions based on the core set of predictive features. The core set of features may provide a floor of essential inputs required to evaluate the treatment prediction model, or may provide a floor of the most desirable or recommended inputs. Depending on the model type, the model may be capable of generating predictions based solely on the core predictive features, and may or may not accept additional inputs related to non-core features. The core predictive features can consist, for example, of a predetermined number n most predictive features, or may be selected on the basis of any feature that exhibits at least a threshold level of predictive power. Since different predictive models are generally trained independently of each other for different treatment modalities, there may be overlap among the core features of each model, but differences may exist as well. In practice, the example study described in this specification found that the most predictive (core) features across many models and treatment modalities can include substantial overlap. The core features utilized across multiple treatment prediction models are referred to herein as “shared” features. Sharing features across multiple predictive models is technically beneficial because it reduces the number of feature values to be derived when evaluating multiple models, and also typically consolidates and reduces the amount of information collected from the patient to generate treatment prediction responses using multiple models. Further, configuring the predictive models to generate predictions on the core feature set rather than requiring values for the full expanded set is useful to reduce the size of the models, thereby reducing model complexity, the amount of storage required by the models, and the computational expense ordinarily involved in evaluating the models.

In some implementations, the system reduces the patient treatment prediction profiles to their core predictive features to provide training inputs, and identifies the target treatment response classifications as the target outputs (310). The treatment prediction model is then trained on the reduced treatment prediction profiles (312). Alternatively, the system trains the model on the expanded feature set initially, and then re-trains or otherwise configures the model to operate on the core predictive features. The model is validated (314), and then placed into production for use in generating predictions for new patients (316).

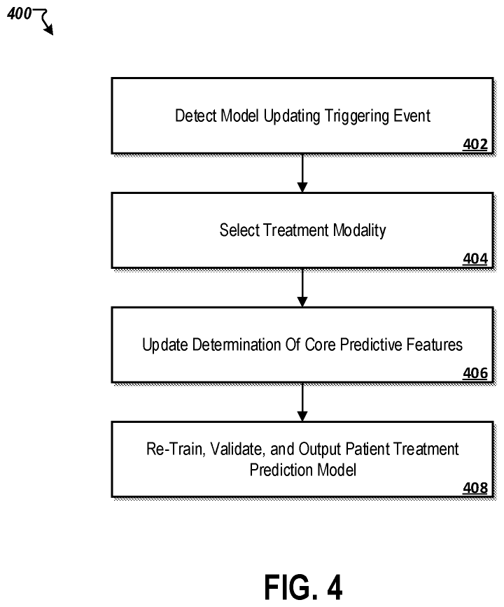

Over time, the analytics system continues to build the treatment prediction profiles database through regular updates and importations of sourced patient data. Additional patient records may become available, data may be collected from new or different sources, and features in the expanded set may change depending on changes in the source data. Therefore, from time to time (e.g., monthly, quarterly, annually), it is beneficial to update the patient treatment prediction models to account for the additions and changes in patient profiles stored in the patient profiles database. FIG. 4 is a flowchart of an example process 400 for updating a patient treatment prediction model in these circumstances. The system (e.g., analytics system 102) detects a model updating triggering event (402). The triggering event may be based on user input requesting immediate updating, or may occur automatically on a scheduled basis (e.g., monthly, quarterly, annually), or based on certain conditions reflecting sufficient changes in the profiles database since the last time the models were updated. The system selects a treatment modality (404), and proceeds substantially as described in process 300 (FIG. 3) to train the predictive model based on the current data available in the profiles database corresponding to the selected treatment modality. Notably, as the expanded features are re-analyzed, the system can update its determination of the core set of predictive features for the model (406). Some or all of the core predictive features may remain unchanged, but other features may be added or dropped from the core set. In this manner, the performance of the updated model can be continuously improved while maintaining a relatively compact model size that does not necessarily retain all core features from a previous training iteration (and does not necessarily include all features in the expanded set). The system accordingly re-trains, validates, and outputs the treatment prediction model as previously discussed (408).

FIG. 5 is a flowchart of an example process 500 for evaluating patient treatment prediction models to generate treatment response predictions. The system receives a patient treatment prediction request (502) from a provider or patient. The request may include sourced patient data, such as EMR/EHR data, PRO data, demographic data, and the like. Portions of the sourced patient data can also be retrieved from other sources (504). The system generates a patient treatment prediction profile (506), and determines values for at least the core predictive features including shared core predictive features (508) and core predictive features that are unique to each model (510). With the core features as input, the system processes the values from these features through the suite of patient treatment prediction models to determine patient treatment prediction responses for various treatment modalities (512). The predictions can be formatted into reports (514), including providing a more detailed provider-facing report (516) and a less detailed patient-facing report (518). The reports are then delivered to the patient and provider through designated channels, such as a web portal (520).

### Example Study: Methods, Results, and Discussion

1. Overview

This section describes an example study conducted in relation to aspects of the techniques disclosed in this specification. The study leveraged patient-reported outcome (PRO) and electronic medical record (EMR)/electronic healthcare record (HER) data from the University of Pittsburgh Medical Center (UPMC) Patient Outcomes Repository for Treatment (PORT) registry to train a machine-learning model (referred to as Personalized Pain Treatment (PPT) model) to predict how likely patients are to respond to 19 common pain medicine interventions such as medications and epidural injections based on their individual phenotypes.

This study was conducted with approval by the University of Pittsburgh Institutional Review Board and the UPMC Quality Improvement Review committee.

2. Source of Data, Participants, and Sample Size

Between Mar. 15, 2016 and Mar. 15, 2020, a total of 6,427 patients with suitable follow-up data attended at least one appointment at one of seven UPMC Pain Medicine specialty clinics in Western Pennsylvania. All patients were treated by one of eight board-certified pain management specialists using a standardized multidimensional assessment and treatment planning approach to maximize the use of multimodal approaches tailored to the needs of the individual patient based on the physician's assessment. Some facets of this multidimensional assessment include not repeating failed treatments and asking patients which treatment(s) would be preferable to them. Evaluation and audits of this standardized comprehensive pain management approach have shown >90% consistency in delivery across all providers and clinics.

Patients completed at least two surveys to self-report pain levels and descriptions at baseline and at 3-month's follow-up for one or more treatments delivered or prescribed on the day of the baseline visit. Treatments administered or prescribed included medications, rehabilitation (physical or occupational therapy), injections, behavioral health care, and/or integrative medicine (e.g., acupuncture). The surveys included validated pain, mental health, and physical health measures from the NIH Patient Reported Outcomes Measurement Information System (PROMIS), the PainDETECT neuropathic pain questionnaire, and impression of change measures to assess global treatment effects.

PRO surveys were considered to be within the baseline time range if they occurred up to 10 days before the first date that the treatment of interest was prescribed, and surveys were considered within the 3-month follow-up time range if they occurred 60-120 days after the baseline treatment date. Follow-up surveys were collected either at a return visit to the clinic or via an emailed survey link. If more than one PRO survey was completed within a baseline time range, the survey closest to the baseline treatment date was used, and for the 3-month follow-up time range, the survey closest to 90 days after the baseline treatment date was used. A total of 26 PRO and 14 EMR domains containing a total of 115 variables were queried for these patients from the UPMC PORT registry, including age, gender, diagnoses, prescribed treatments, and outcome measures. A list of the variables used to train the PPT algorithm are listed in Table 5. Demographic information and baseline PRO measures for these patients are shown in Table 1.

3. Data Preparation and Missing Data

All identifiable patient variables, including medical record numbers, were encrypted before analysis. Custom variables were created for derived features to flag specific treatments and diagnoses, calculate summary scores such as the Charlson Comorbidity Index for medical comorbidities, and to organize and optimize data for analysis (see Table 5). Missing values in discrete variables were replaced with a null category, and missing values in continuous variables were replaced with the mean of the non-missing values.

4. Outcomes

The outcome analyzed for prediction of treatment response in 18 of the 19 pain treatment datasets was a combined benchmark for a clinically meaningful response at 3 months (+/−30 days) after the first prescription/performance date of the chronic pain treatment of interest. Using benchmarks for pain treatment success, a patient needed to meet at least one of the following thresholds at 3-month's follow-up to be considered a treatment responder: (1) ≥30% improvement in average pain intensity on a 0-10 numeric scale over the last 7 days, (2) ≥5 points improvement in the PROMIS Physical Function T-Score, and/or (3) a report of “Very Much Improved” (the highest patient-rated category of improvement) on the overall Impression of Change scale. The exception was the Behavioral Medicine dataset, where the responder outcome analyzed was a combined response benchmark of minimal clinically important differences of either (1) ≥20% improvement in average pain intensity, (2) ≥2 points improvement in physical function, and/or (3) a report of “Slightly Improved” or “Very Much Improved” on the overall Impression of Change scale.

5. Predictors and Statistical Analysis Methods

The tree-based random forest ensemble learning method was applied to 19 separate datasets. Random forest utilizes a multitude of decision trees superimposed on regression to select independent variables. It has the advantage of reducing model overfitting compared with standard regression approaches. These datasets represent common categories of chronic pain treatment (Table 2) including receiving and/or being prescribed any kind of multimodal pain treatment approach (omnibus category), as well as receiving and/or being prescribed specific treatments such as anticonvulsant medication, epidural steroid injections, rehabilitation therapy, and behavioral medicine. The specific medications and procedure codes included in each treatment category are listed in Table 6. A total of 40 PRO and EMR domain variables were used to train these models (Table 5). As each treatment model was trained separately, some patients appeared in the training datasets for more than one model if they had been prescribed more than one treatment (on one or more occasions) and had completed PRO surveys corresponding to each treatment's baseline and 3-month follow-up time ranges. Data preparation and manipulation was performed via Python using the pandas and NumPy software libraries.

All random forest models were trained in Python using the scikit-learn software library. The tuning parameters used in the random forest models were the number of trees, the number of features to consider at every split, the maximum number of levels in a tree, the minimum number of samples required at each leaf node, and the minimum number of samples required to split a node. Each of the 19 treatment datasets containing patient treatment profiles was randomly split into a 70% training set and a 30% testing set. A 5-fold cross-validation was applied during the training process to select the best combination of parameters in random forests. The trained random forest models generated probabilities that a specific treatment would result in a patient reaching the combined benchmark for clinically meaningful response (detailed above) based on each patient's unique phenotype of PRO+EMR variables. To calculate a prediction probability for a new patient in the testing set, the majority response outcome was used, and the predicted probability was calculated as the number of trees generating the response over the total number of trees. Once the predicted probabilities were obtained, the Area Under the Receiver Operating Characteristics curve (AUROC) and expected calibration error (ECE) were calculated as measures of model performance.

5. Model Tuning and Feature Selection

Treatment models with an AUROC ≥0.65 were considered to be performing sufficiently well, as an AUROC at or above this threshold is an acceptable benchmark for statistical models to predict treatment responses related to pain management. The technique of selective AUROC (SAUROC), which is a variant of a partial AUROC approach, was applied to treatment models not meeting this threshold in the initial random forest modeling to reach a dataset fraction with SAUROC ≥0.65. The predicted probabilities of treatment response were sorted in descending order, then data were examined iteratively to report the SAUROC values of the patient data in upper and lower thresholds of the dataset. Finally, the highest SAUROC and threshold levels that used at least 75% of the data and 67% of the data were recorded. A probability calibration technique was applied to the remaining treatment models still not meeting the required threshold of SAUROC ≥0.65 using ≥75% or 67% of available data. Probability calibrations applied either the Platt's sigmoid method or the isotonic approach with 5-fold cross-validation to calibrate the random forest classifier and report probabilities of treatment response based on the calibrated models.

In addition to having AUROC/SAUROC ≥0.65, all models had to have a calibration error≤0.25 to be considered to be performing acceptably. A calibration error of 0.25 indicates that up to 25% of the time, the predicted treatment outcome generated by the random forest model is different than the actual treatment outcome.

Feature importance scores were calculated for each predictive variable using the mean decrease in Gini index, a measure of total variance across the two classes of response or non-response. Larger decreases in Gini index indicate features that are more predictive and more important to the performance in the random forest model.

6. Patient Outcomes and Model Outputs

Table 1 displays the demographics and baseline clinical features of the patient cohort (n=6427). To summarize, the average age was 57.5 years, 62% were female, 85% were Caucasian, and the educational level of a majority of patients was a high school diploma. Spinal pain was the most common pain complaint (66.7%), followed by arthritis, neuropathic, and fibromyalgia pain, respectively. The mean duration of pain was 7.14 years with a median of 3.1 years; in other words, half of all subjects had experienced pain for less than 3.1 years. Other notable features include a baseline average pain intensity rating of 6.4/10, high levels of pain interference (mean T-Score=66.2), poor self-reported functioning (mean T-Score=35.3), and elevated levels of depression, anxiety, and sleep disturbance symptoms compared to the general population. As a whole, this is a broad patient population with a range of pain syndromes and significant variability in pain, function, sleep, and mental health symptoms across the cohort. Such diversity in clinical presentations is a necessary precursor for high quality predictive modeling to be clinically useful. Table 2 shows the response rates of patients used to train the 19 random forest pain treatment models and the ranges of responding probabilities that each trained model will produce for new patient data. Overall, 42% of patients receiving multimodal pain treatment were treatment responders and showed clinically meaningful and significant improvement within 3 months, regardless of which treatment was administered (Multimodal Pain Treatment dataset). The response rates for the specific categories of pain treatment using the higher (i.e., ≥30% improvement in pain intensity) benchmark for treatment response ranged from 28% for intrathecal pain pumps and implanted devices (such as spinal cord stimulators) to 46% for naproxen NSAID medication. The response rate for behavioral medicine, which used a lower combined response benchmark (i.e., ≥20% improvement in pain intensity) was 57%. Across these 19 treatment categories, the range of predicted probabilities of response (derived from patient phenotypes) was 21-87%.

7. Model Performance

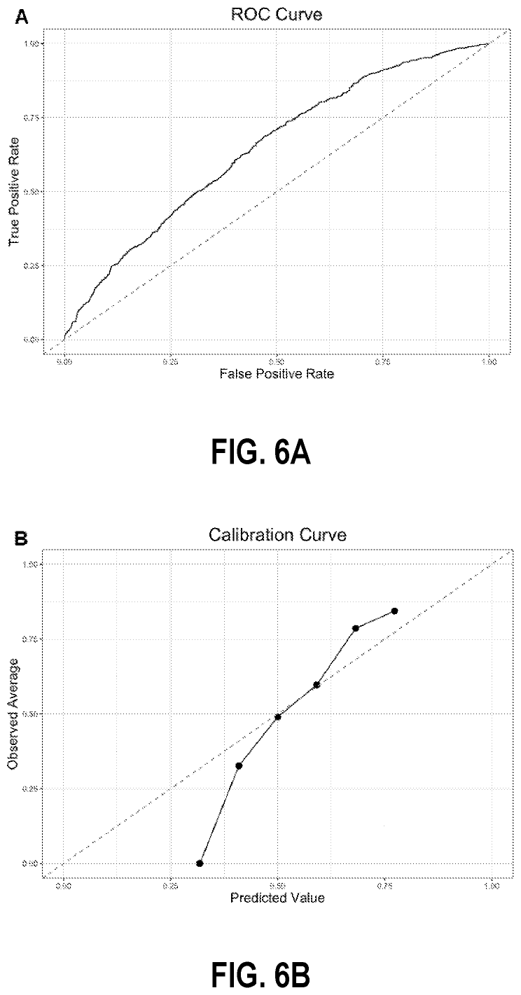

Table 3 shows the AUROC and the methodology used to obtain those values, such as using all or a portion of the dataset to reach an AUROC ≥0.65. Two of the 19 treatment models, multimodal pain management and antidepressant medications, met the model performance thresholds of AUROC ≥0.65 and calibration error≤0.25 using all available data. FIGS. 6A-6B shows example AUROC and calibration curves for the multimodal pain management model. Applying the selective AUROC technique with or without probability calibration resulted in 17 more treatment models reaching these model performance thresholds using either ≥75% or ≥67% of the patient data. Expected calibration errors for the 19 treatment models ranged from 0.03 for medications of any type to 0.23 for integrative medicine. The average calibration error was 11%, which is the frequency that the predicted outcome was different than the actual outcome in the data set.

It is noted that, in this example study, the range of predicted probabilities of response produced by a trained random forest model has little correlation to the response rate for the patient population as a whole in the training dataset, especially when the selective AUROC and probability calibration techniques are applied to a subset of the patient population. For example, patients in the training dataset for the treatment category of intrathecal pain pumps and implanted devices showed a low response rate of 28%, but the range of predicted probabilities of response for this trained model is 60%-83%. The random forest model for this pain treatment category had a selective AUROC of 0.65 using at least 67% of the available patient data and sigmoid probability calibration. When new patient data are applied to this trained model, approximately ⅔ of patients will be given a predicted probability of response in the range of 60%-83%, but ⅓ of patients cannot be given a prediction with that level of accuracy. The default reporting for patients in the latter category is “no valid prediction possible.”

8. Predictive Features

Table 4 shows the variables that had the highest predictive validity across the 19 treatment categories based on their feature importance scores, along with the ranges of these scores and the directionality and categories associated with a clinically significant response. This cluster of highly predictive variables was remarkably consistent across all of the 19 treatments (see Tables 7 and 8 and discussed in more detail below). All highly predictive variables in this study were obtained from PRO variables or patient demographics, such as age and gender. In other words, EMR variables such as body mass index, tobacco use, or medical comorbidities, did not significantly predict treatment responses for any of the 19 treatments. For continuous variables such as age, anxiety, and depression, the direction of the response was consistent for all treatment model training datasets with a significant mean difference between responding and non-responding patients (p <0.05). For example, responding patients in the multimodal pain treatment, medications (any type), anticonvulsant medications, antidepressant medications, celecoxib NSAID medication, and integrative medicine training datasets showed mean (SD) ages of 55.5 (13.9) to 62.0 (12.4), whereas non-responding patients in these datasets showed mean (SD) ages of 53.6 (13.3) to 58.5 (13.2), p's=0.001-0.040. In all cases the mean age of the responding patients was higher (i.e., older) than the mean age of the non-responding patients. The unadjusted associations between the candidate predictor variables are summarized in Table 4. List of the specific pain treatment categories that showed significant difference (p<0.05) between responding and non-responding patients is shown in Table 7 for continuous variables/features and Table 8 for categorical variables/features.

9. Discussion

This study demonstrates that the response to many common treatments for chronic pain can be reliably and accurately predicted based on a patient's presenting phenotype, as represented in a patient treatment profile. Using machine-learning model, and in particular random forest generated models in this study, patient reported outcomes and basic demographic data for approximately 12 common domains were capable of being evaluated with the treatment prediction models to predict likelihoods/probabilities that patients would respond to each of 19 typical treatments. The models can thus address key clinical practice gaps concerning the lack of available tools to help individual patients and clinicians decide which treatments for chronic pain should be prescribed.

This technology implicates several clinical applications. With further development into a shared decision-making software platform, patients and providers can use pain treatment prediction models to agree on treatment approaches to managing chronic pain, with possibly better clinical outcomes, since the treatments more likely to be effective would typically be most preferred. Getting the right patients to the right treatments more quickly may also reduce healthcare costs (through avoiding trials of failed treatments for example), and thus directly improve value-based pain care, i.e., improving clinical outcomes and reducing costs simultaneously. Rigorous collection and use of patient reported outcomes are a key necessary driver for transitioning to value-based healthcare in the United States.

Major strengths of the medical informatics platform (PORT) underpinning this study included: (1) Use of high quality, comprehensive, and valid outcome measures in the field of pain management, (2) Collection of those outcome measures at regular intervals after the initiation of treatment, and (3) Cross-linking of treatment selection, patient reported outcomes, and EMR variables at each visit. A uniform assessment and multimodal treatment planning approach amongst the providers in this study also promotes clinically valid predictive modeling. As a whole, these approaches enhance the scientific rigor of the study and provide greater credibility to the findings.

The pain syndrome characteristics of the patient population in this study were, as a whole, quite broad, such as significant variability in the average duration of pain, and levels of functional impairment. The average response rate to any of the treatments was 42% and it is remarkable that a high rate of significant improvement was obtained for treatment of chronic painful conditions. Of course, the providers are selecting treatments which in their clinical judgment are most likely to work in any particular patient. Such “dynamic range” in the predictor variables and response rates lends itself to better and more valid machine learning based modeling. That is, if phenotypes in the clinical population were homogenous and if all the patients got better, the models are unlikely to be useful clinically, even if the AUROC's were >0.95. The findings in this study indicate that the heterogeneity of phenotypes and treatment outcomes in the sample population yielded dynamic ranges of treatment response probabilities from 21-87%. In other words, the models can indicate to patients and providers which treatments are most likely and least likely to work for them, which clinically is very salient.

The directionality of the core/highest predictive features was also very consistent across all of the 19 models. For example, older patients who may develop chronic pain as a part of normal aging are more likely to respond to treatments, whereas younger patients with higher negative affect at baseline are less likely to respond. One of the implications for development of the models is that these core predictors can be obtained easily from patients through brief, validated self-report measures.

Certain variables in this study were not predictive of outcome, including race, BMI, and socioeconomic status (as measured by Medicaid insurance as proxy). Disparities in health care and in pain care outcomes specifically are well-documented in subpopulations who are not white, have high BMI, and/or with low socioeconomic status. Yet the results in this study indicate that when such populations are offered high-quality pain treatment, they are just as likely to respond. Thus, our findings imply that disparities relate to chronic pain treatment access more so than treatment outcomes.

Another implication of findings in this study is that in predicting pain treatment outcomes successfully, the physical examination may not be as important, since despite not having this information the models performed well and appear clinically useful. In an era where it is increasingly relevant to optimize virtual/remote pain care (i.e., telemedicine), if the physical exam is indeed less important to selecting an effective treatment, then digital tools such as patient treatment prediction models are even more relevant and may enhance the delivery of remote care.

Other possible study limitations center on the issues of external validity. Although diverse in clinical presentations, the patient population was primarily from Western Pennsylvania. In addition, the outcomes were tracked in patients receiving treatment from pain specialists only and not from primary care physicians or other specialists. Although, compared to a primary care physician the only unique treatment pain specialists perform and offer are the injections, and the prescribing of medications and physical therapy (for example) is largely the same. Thus, the applicability of the treatment prediction models to primary care settings may still be high, as those providers deliver the majority of pain care throughout the United States.

10. Tables

TABLE 1
Demographics, pain characteristics, and baseline outcome measures
for chronic pain patients receiving care at the UPMC Pain Medicine
clinics between March 2016 and March 2020 whose PRO and EMR data
were used to train the predictive models in the example study.

| Variable | Patients (N = 6427) |
|---|---|
| Patient characteristics | |
| Age (Mean (SD)) | 57.5 (15.2) |
| Sex, Female (%) | 61.8% |
| Race (%) | |
| Black | 12.8% |
| White | 85.2% |
| Other races | 1.60% |
| Declined/Not specified | 0.40% |
| Education, Highest Level Completed (%) | |
| Less than high school | 1.00% |
| High school diploma | 40.5% |
| College degree | 32.3% |
| Medicaid Insurance, Yes (%) | 17.9% |
| Comorbidity (Mean (SD))¹ | 0.48 (0.86) |
| Tobacco Use, Yes (%) | 21.4% |
| Alcohol Use, Yes (%) | 46.4% |
| Illicit Drug Use, Yes (%) | 11.5% |
| Pain characteristics | |
| Pain Experience Duration, Years (Mean (SD), Median) | 7.14 (9.38), 3.08 |
| ICD10 Diagnoses (%) | |
| Back, neck, and spine pain | 66.7% |
| Low back pain | 57.5% |
| Fibromyalgia | 5.70% |
| Migraine | 0.60% |
| Neuropathic pain and nerve injuries | 14.2% |
| Osteoarthritis | 3.80% |
| Other arthritis and arthropathy | 8.90% |
| Other chronic pain conditions | 33.8% |
| Other musculoskeletal pain | 5.10% |
| Rheumatoid arthritis | 0.50% |
| Patient-reported outcomes at baseline (Mean (SD)) | |
| Regions selected on pain body map (0-74) | 10.8 (10.8) |
| Pain intensity, average in last 7 days (0-10)² | 6.41 (1.99) |
| Pain interference (mean (SD) T-Score, 0-100)² | 66.2 (6.19) |
| Pain behavior (mean (SD) T-Score, 0-100)² | 60.8 (3.02) |
| Neuropathic pain score (0-3 8)³ | 14.8 (8.41) |
| PROMIS Physical function (mean (SD) T-Score, 0-100)² | 35.3 (7.01) |
| PROMIS Global physical health (mean (SD) T-Score, 0-100)⁴ | 33.7 (6.21) |
| PROMIS Anxiety (mean (SD) T-Score, 0-100)² | 55.8 (9.89) |
| PROMIS Depression (mean (SD) T-Score, 0-100)² | 54.9 (10.3) |
| PROMIS Sleep disturbance (mean (SD) T-Score, 0-100)² | 59.4 (9.20) |
| PROMIS Global mental health (mean (SD) T-Score, 0-100)⁴ | 44.2 (7.83) |
| Positive Outlook (mean (SD) T-Score, 0-100)⁵ | 48.4 (10.1) |

TABLE 2
Patient response rates and ranges of responding probabilities
for 19 chronic pain treatment categories.

| | Number of | Number of | Response | Range of Responding | |
|---|---|---|---|---|---|
| Type of Treatment | Patients | Responders | Rate | Probabilities | |
| Multimodal Pain Management | 6308 | 2678 | 0.42 | 0.43 | 0.77 |
| Medications - Any Type Below | 4984 | 2003 | 0.40 | 0.44 | 0.75 |
| Naproxen Medication | 216 | 99 | 0.46 | 0.31 | 0.75 |
| Schedule III/IV Opioid Medications | 1374 | 575 | 0.42 | 0.45 | 0.73 |
| Anticonvulsant Medications | 2983 | 1160 | 0.39 | 0.43 | 0.76 |
| Muscle Relaxant Medications | 2271 | 872 | 0.38 | 0.46 | 0.75 |
| Meloxicam Medication | 714 | 272 | 0.38 | 0.29 | 0.84 |
| Aspirin Medication | 171 | 65 | 0.38 | 0.21 | 0.87 |
| Celecoxib Medication | 399 | 146 | 0.37 | 0.36 | 0.84 |
| Antidepressant Medications | 1663 | 574 | 0.35 | 0.40 | 0.77 |
| Cervical Epidural Injections | 300 | 131 | 0.44 | 0.36 | 0.66 |
| Interlaminar Lumbar Epidural Injections | 1807 | 761 | 0.42 | 0.39 | 0.78 |
| Muscle and Tendon Injections | 474 | 196 | 0.41 | 0.41 | 0.75 |
| Abdominal and Pelvic Blocks | 109 | 45 | 0.41 | 0.41 | 0.77 |
| Pain Pumps and Other Implanted Devices | 190 | 54 | 0.28 | 0.60 | 0.83 |
| Behavioral Medicine | 275 | 158 | 0.57 | 0.17 | 0.52 |
| Integrative Medicine | 172 | 75 | 0.44 | 0.47 | 0.68 |
| Rehabilitation Therapies | 572 | 202 | 0.35 | 0.49 | 0.72 |
| Orthotics and Assistive Devices | 133 | 45 | 0.34 | 0.52 | 0.76 |

TABLE 3
Area under the Receiver Operating Characteristics curve (AUROC) for random
forest models trained to predict response to common treatments for chronic
pain and the methodology used to obtain those AUROC values.

| | | Expected | |
|---|---|---|---|
| | AUROC | Calibration | Random Forest |
| Type of Treatment | (95% C.I.) | Error | Methodology |
| Multimodal Pain Management | 0.65 (0.62, 0.67) | 0.04 | Standard AUROC |
| Antidepressant Medications | 0.66 (0.61, 0.71) | 0.05 | using all available data |
| Aspirin Medications | 0.69 (0.49, 0.84) | 0.18 | Selective AUROC |
| Muscle and Tendon Injections | 0.69 (0.58, 0.79) | 0.10 | using >75% of data |
| Schedule III/IV Opioid Medications | 0.68 (0.62, 0.74) | 0.07 | |
| Cervical Epidural Injections | 0.67 (0.55, 0.78) | 0.09 | |
| Anticonvulsant Medications | 0.65 (0.61, 0.69) | 0.05 | |
| Medications - Any Type Listed | 0.65 (0.62, 0.69) | 0.03 | |
| Meloxicam Medication | 0.65 (0.57, 0.74) | 0.06 | |
| Muscle Relaxant Medications | 0.65 (0.60, 0.69) | 0.04 | |
| Naproxen Medication | 0.65 (0.48, 0.79) | 0.12 | |
| Rehabilitation Therapies | 0.65 (0.56, 0.73) | 0.09 | Selective AUROC |
| Interlaminar Lumbar Epidural Injections | 0.65 (0.60, 0.70) | 0.07 | using >75% of data |
| | | | and isotonic correction |
| Orthotics and Assistive Devices | 0.70 (0.51, 0.86) | 0.15 | Selective AUROC |
| Behavioral Medicine | 0.66 (0.52, 0.78) | 0.18 | using >75% of data |
| | | | and sigmoid correction |
| Celecoxib Medication | 0.65 (0.51, 0.76) | 0.15 | Selective AUROC |
| | | | using >67% of data |
| Abdominal and Pelvic Blocks | 0.66 (0.41, 0.90) | 0.21 | Selective AUROC |
| Pain Pumps and Other Implanted Devices | 0.65 (0.43, 0.83) | 0.15 | using >67% of data |
| Integrative Medicine | 0.65 (0.44, 0.86) | 0.23 | and sigmoid correction |

TABLE 4
Variables showing the highest predictive validity in the random forest models trained for the 19 chronic pain treatment categories and
subcategories. For continuous variables, directionality associated with a positive response was determined by comparing the means of
responding to non-responding patients when the difference between the two patient groups was statistically significant (p < 0.05).

| | Range of | | | | Direction/Category |
|---|---|---|---|---|---|
| | Feature | Range of Means (SD) | Range of Means (SD) | Ranges of | Associated with |
| Feature (at Baseline) | Scores | of Non-Responders | of Responders | P-Values | Positive Response |
| Continuous variables: | | | | | |
| Age, years | 0.026-0.052 | 53.6 (13.3)-58.5 (13.2) | 55.5 (13.9)-62.0 (12.4) | 0.000-0.040 | Older patients |
| Anxiety T-Score¹ | 0.023-0.065 | 56.1 (9.30)-57.3 (10.0) | 52.8 (10.8)-55.9 (10.3) | 0.000-0.047 | Lower anxiety |
| Depression T-Score¹ | 0.028-0.052 | 52.9 (10.5)-56.3 (10.1) | 51.5 (10.4)-54.8 (10.6) | 0.000-0.033 | Lower depression |
| Global Mental Health T-Score² | 0.022-0.044 | 40.6 (7.56)-45.0 (7.52) | 41.5 (8.20)-47.1 (7.23) | 0.000-0.029 | Higher global mental health |
| Global Physical Health T-Score² | 0.021-0.045 | 32.5 (5.28)-33.8 (5.00) | 33.5 (6.40)-35.2 (7.49) | 0.000-0.045 | Higher global physical health |
| Number of Body Map Regions | 0.023-0.044 | 9.28 (8.66)-14.0 (12.9) | 7.81 (7.29)-11.6 (12.0) | 0.000-0.017 | Fewer regions selected |
| Pain Behavior T-Score¹ | 0.025-0.055 | 61.0 (2.99)-61.6 (2.14) | 60.7 (3.04)-61.4 (2.38) | 0.000-0.031 | Lower pain behavior |
| Pain Experience Duration, years | 0.008-0.022 | 5.82 (1.92)-7.93 (5.16) | 5.28 (1.40)-7.00 (4.67) | 0.003-0.042 | Less pain duration |
| Pain Interference T-Score¹ | 0.025-0.049 | 66.4 (5.97)-66.5 (5.94) | 66.0 (6.46)-66.0 (6.48) | 0.014-0.018 | Lower pain interference |
| Neuropathic Pain Score³ | 0.027-0.056 | 15.6 (8.53)-18.1 (8.20) | 13.3 (8.59)-15.7 (8.75) | 0.000-0.046 | Lower neuropathic pain |
| Positive Outlook T-Score⁴ | 0.015-0.025 | 41.3 (5.00)-50.4 (4.79) | 42.4 (4.50)-51.8 (5.16) | 0.000-0.044 | Higher positive outlook |
| Sleep Disturbance T-Score¹ | 0.030-0.065 | 57.7 (8.99)-61.1 (8.46) | 55.6 (10.1)-60.0 (9.42) | 0.000-0.044 | Lower sleep disturbance |
| Categorical variables: | | | | | |
| Prescription opioid user, little or no | 0.015-0.032 | 84.9%-88.8% | 74.9%-77.2% | <0.001 | Fewer opioid users |
| misuse⁵, % | | | | | |

TABLE 5
Patient-reported outcome (PRO) and electronic medical record (EMR)
variables used to train the PPT predictive analytical algorithm.

| Variable | Source | Custom Variable Source |
|---|---|---|
| Outcome measures | | |
| Pain intensity, % improvement | PRO | Pain intensity, average in last 7 days |
| Physical function, change in T-Score | PRO | PROMIS Physical Function T-Score |
| Impression of change | PRO | |
| Patient characteristics | | |
| Age | EMR | |
| Sex | EMR | |
| Race | EMR | |
| Education, highest level completed | PRO | |
| Medicaid insurance | EMR | Visit financial class |
| Charlson Comorbidity Index | EMR | Encounter diagnoses, problem list |
| Tobacco use | EMR | |
| Alcohol use | EMR | |
| Illicit drug use | EMR | |
| Involved in legal action related to pain | PRO | |
| Work status | PRO | |
| Disability assistance status | PRO | |
| Marital status | PRO | |
| Living with a significant other | PRO | |
| State of residence | EMR | Zip code, US Census API |
| County of residence | EMR | Zip code, US Census API |
| Pain characteristics and measures | | |
| Number of regions selected on the pain body map | PRO | Pain body map |
| Pain experience duration | PRO | |
| Experienced chronic pain as a child | PRO | |
| PainDETECT neuropathic pain score | PRO | |
| Body map cluster membership | PRO | Pain body map |
| Diagnosis in ICD10 categories present in at least | EMR | Encounter diagnoses |
| 5% of the patient population (F11, G89, M25, M47, | | |
| M48, M50, M51, M54, M79, M96, R10, Z79) | | |
| Chronic pain diagnoses: | EMR | Encounter diagnoses and custom-made |
| Back, neck or spine pain | | diagnosis look-up tables |
| Low back pain | | |
| Neuropathic pain or nerve injuries | | |
| Fibromyalgia | | |
| Migraine | | |
| Osteoarthritis | | |
| Rheumatoid arthritis | | |
| Other arthritis or arthopathy | | |
| Other musculoskeletal pain | | |
| Other chronic pain conditions | | |
| Presence of pain in major anatomical regions | PRO | Pain body map |
| (abdomen, ankle, arm, buttocks, chest, elbow, foot, | | |
| hand, head, hip, knee, leg, lower back, neck, | | |
| pelvis/groin, shoulder, upper back, wrist) | | |
| PROMIS Pain Behavior T-Score | PRO | |
| PROMIS Pain Interference T-Score | PRO | |
| Mental health measures | | |
| Post-traumatic stress disorder score | PRO | |
| Opioid Misuse Index | PRO | |
| HEAL Positive Outlook T-Score | PRO | |
| History of psychiatric hospitalization | PRO | |
| PROMIS Anxiety T-Score | PRO | |
| PROMIS Depression T-Score | PRO | |
| PROMIS Global Mental Health T-Score | PRO | |
| Physical function measures | | |
| PROMIS Global Physical Health T-Score | PRO | |
| PROMIS Sleep Disturbance T-Score | PRO | |
| Other health-related measures | | |
| Common co-morbid diagnoses: | EMR | Encounter diagnoses, problem list, and |
| Anxiety | | custom-made diagnosis look-up tables |
| Congestive heart failure | | |
| Connective tissue disorder | | |
| Depression | | |
| Diabetes | | |
| Hypothyroidism | | |
| Irritable bowel syndrome | | |
| PTSD | | |
| Seizure disorders | | |
| Sleep apnea | | |
| Thyroid disorders | | |
| Concurrent medication use: | EMR | Medication orders and custom-made |
| Schedule II/III/IV opioids | | medication look-up tables |
| Antidepressants | | |
| NSAIDs | | |
| Muscle relaxants | | |

TABLE 6
Medication and procedure orders prescribed in UPMC Pain Medicine clinics between
Mar. 15, 2016 and Mar. 15, 2020 that were included in pain treatment categories.

| Pain Treatment Category | Specific Medication/Procedure Orders |
|---|---|
| Multimodal Pain Management | Any medication or procedure order from a UPMC Pain Medicine clinic |
| Medications - Any Type | Any medication targeting pain, mood, or physical function ordered in a UPMC Pain Medicine clinic |
| Anticonvulsant Medications | Acetazolamide 125, 250 mg tablet |
| | Acetazolamide ER 500 mg (diamox) 12 hr capsule |
| | Amantadine 8% gabapentin 6% lidocaine 5% cream |
| | Amantadine8 clonidine0.2 gabapentin6 amitriptyline3 tetracaine2 |
| | Carbamazepine 100 mg chewable tablet |
| | Carbamazepine 100 mg/5 ml oral suspension |
| | Carbamazepine 200 mg tablet, Carbamazepine oral |
| | Carbamazepine ER 100 mg (tegretol xr) 12 hr tablet |
| | Carbamazepine ER 200, 300 mg capsule, extended release mphase12 hr |
| | Carbamazepine er 200, 400 mg tablet, extended release, 12 hr |
| | Depakote ER oral |
| | Depakote oral |
| | Dilantin extended 100 mg capsule, Dilantin oral |
| | Divalproex 125 mg capsule, delayed release sprinkle |
| | Divalproex 125, 250, 500 mg tablet, delayed release |
| | Divalproex ER 250, 500 mg tablet, extended release 24 hr |
| | Divalproex oral |
| | Gabapentin (bulk) 100% powder |
| | Gabapentin (bulk) misc |
| | Gabapentin 100 mg capsule |
| | Gabapentin 25 mg/ml oral suspension |
| | Gabapentin 250 mg/5 ml (5 ml), 300 mg/6 ml (6 ml) oral solution |
| | Gabapentin 250 mg/5 ml oral solution |
| | Gabapentin 300, 400 mg capsule, Gabapentin 600, 800 mg tablet, |
| | Gabapentin oral |
| | Gabapentin enacarbil ER 300 mg (horizant) tablet |
| | Gabapentin enacarbil ER 600 mg tablet, extended release |
| | Gabapentin enacarbil oral |
| | Gabapentin ER 300 mg (9)-600 mg (69) tablet, extended release 24 hr |
| | Gabapentin ER 300, 600 mg tablet, extended release 24 hr |
| | Gabapentin-diet. Supp 11 oral |
| | Gralise 600 mg tablet, extended release |
| | Horizant er 300, 600 mg tablet, extended release |
| | Keppra oral |
| | Ketamine 10% gabapentin 6% lidocaine 5% cream |
| | Ketamine 10 baclofen2 cyclobenz2 diclofenac3 gabapentin6 tetracaine2 |
| | Ketamine 10 baclofen2 gabapentin6 amitriptyline3 nifedipine2 tetracaine |
| | Ketamine 10% baclofen2% cyclobenz2% gabapentin6% lido5% tetracaine2% |
| | Ketamine 10% clonidine0.2% gabapentin6% amitriptyline3% tetracaine2% |
| | Ketoprofen 10% lidocaine 3% gabapentin 6% cream |
| | Lacosamide 50, 100, 150, 200 mg tablet |
| | Lamictal 25 mg tablet, Lamictal oral |
| | Lamictal starter (orange) kit 25 mg (42)-100 mg (7) tablets, dose pack |
| | Lamotrigine 50, 100, 200 mg disintegrating tablet |
| | Lamotrigine 25, 100, 150, 200 mg tablet, Lamotrigine oral |
| | Lamotrigine 25 mg chewable dispersible tablet |
| | Lamotrigine ER 50, 200 mg tablet, extended release 24 hr |
| | Levetiracetam 250, 500, 750, 1,000 mg tablet |
| | Levetiracetam 100 mg/ml oral solution |
| | Levetiracetam ER 500 mg (keppra xr) 24 hr tablet |
| | Levetiracetam ER 750 mg tablet, extended release 24 hr |
| | Lyrica 25, 50, 75, 100, 150, 200, 225, 300 mg capsule, Lyrica oral |
| | Mysoline 250 mg tablet |
| | Neurontin 100, 300, 400 mg capsule, Neurontin 600, 800 mg tablet, Neurontin oral |
| | Neuropathic and anti-inflammatory cream group 2 |
| | Neuropathic pain compound cream kagt |
| | Neuropathic pain compounded cream anl |
| | Neuropathic pain compounded cream kbcgl |
| | Neuropathic pain cream with ketamine |
| | Neuropathy cream - standard neuropathic |
| | Oxcarbazepine 150, 300, 600 mg tablet |
| | Oxcarbazepine ER 300 mg tablet, extended release 24 hr |
| | Oxtellar XR 600 mg tablet, extended release |
| | Phenobarb-hyoscyamn-atropine-scop 16.2 mg-0.1037 mg-0.0194 mg tablet |
| | Phenobarbital 16.2, 32.4, 60, 64.8 mg tablet |
| | Phenobarbital 20 mg/5 ml (4 mg/ml) oral elixir |
| | Phenytoin 125 mg/5 ml oral suspension |
| | Phenytoin 50 mg chewable tablet |
| | Phenytoin sodium extended 100, 200, 300 mg capsule |
| | Pregabalin 100, 150 mg capsule |
| | Pregabalin 20 mg/ml oral solution |
| | Pregabalin 25, 50, 75, 200, 225, 300 mg capsule |
| | Primidone 50, 250 mg tablet, Primidone oral |
| | Tegretol oral |
| | Tegretol XR 200, 400 mg tablet, extended release, Tegretol XR oral |
| | Tiagabine 4 mg tablet |
| | Topamax 15 mg sprinkle capsule |
| | Topamax 50, 100 mg tablet, Topamax oral |
| | Topiramate 100, 200 mg tablet |
| | Topiramate 15, 25 mg sprinkle capsule |
| | Topiramate 25, 50 mg tablet |
| | Topiramate XR 50, 100 mg capsule, extended release 24 hr |
| | Trileptal oral |
| | Valproic acid (as sodium salt) 250 mg/5 ml oral solution |
| | Valproic acid 250 mg capsule |
| | Vimpat 50, 150, 200 mg tablet |
| | Zonisamide 25, 50, 100 mg capsule |
| Antidepressant Medications | Amitriptyline 10, 25, 50, 75, 100, 150 mg tablet, Amitriptyline oral |
| | Amoxapine 25, 100 mg tablet |
| | Buproban oral |
| | Bupropion Hcl 75, 100 mg tablet, Bupropion Hcl oral |
| | Bupropion Hcl 150 mg tablet, 12 hr sustained-release(smoking deterrent) |
| | Bupropion Hcl SR 100, 150, 200 mg tablet, 12 hr sustained-release |
| | Bupropion Hcl XL 150, 300, 450 mg 24 hr tablet, extended release |
| | Celexa oral |
| | Citalopram 10, 20, 40 mg tablet, Citalopram oral |
| | Clomipramine 25, 50, 75 mg capsule |
| | Cymbalta 20, 60 mg capsule, delayed release, Cymbalta oral |
| | Desipramine 10, 25, 50, 75, 100 mg tablet |
| | Desvenlafaxine ER 50, 100 mg tablet, extended release 24 hr |
| | Desvenlafaxine fumarate ER 100 mg tablet, extended release 24 hr |
| | Desvenlafaxine succinate ER 100 mg (pristiq) 24 hr tablet |
| | Desvenlafaxine succinate ER 25, 50 mg tablet, extended release 24 hr |
| | Desyrel oral |
| | Doxepin 10, 25, 50, 75, 100, 150 mg capsule, Doxepin 3, 6 mg tablet, Doxepin oral |
| | Doxepin 5% topical cream |
| | Doxepin Hcl (bulk) powder |
| | Duloxetine 20, 30, 40, 60 mg capsule, delayed release, Duloxetine oral |
| | Effexor oral |
| | Effexor XR 75, 150 mg capsule, extended release, Effexor XR oral |
| | Escitalopram 5, 10, 20 mg tablet |
| | Escitalopram oxalate oral |
| | Fetzima 20, 40, 80, 120 mg capsule, extended release |
| | Fluoxetine 10, 20, 40 mg capsule, Fluoxetine 10, 20, 60 mg tablet, Fluoxetine oral |
| | Fluvoxamine 25, 50, 100 mg tablet, Fluvoxamine oral |
| | Fluvoxamine ER 100 mg capsule, extended release 24 hr |
| | Imipramine 10, 25, 50 mg tablet |
| | Imipramine pamoate 100, 125 mg capsule |
| | Levomilnacipran ER 20, 40, 80, 120 mg capsule, 24 hr, extended release |
| | Lexapro oral |
| | Maprotiline 75 mg tablet |
| | Milnacipran 12.5 mg (5)-25 mg(8)-50 mg(42) tablets in a dose pack |
| | Milnacipran 12.5, 25, 50, 100 mg tablet |
| | Mirtazapine (bulk) 100% powder |
| | Mirtazapine 15, 30 mg disintegrating tablet |
| | Mirtazapine 7.5, 15, 30, 45 mg tablet, Mirtazapine oral |
| | Nefazodone 200 mg tablet |
| | Nortriptyline 10, 25, 50, 75 mg capsule, Nortriptyline oral |
| | Nortriptyline 10 mg/5 ml oral solution |
| | Pamelor oral |
| | Paroxetine 10, 20, 30, 40 mg tablet |
| | Paroxetine ER 25, 37.5 mg tablet, extended release 24 hr |
| | Paroxetine mesylate (menopausal symptoms suppressant) 7.5 mg capsule |
| | Paxil 20 mg tablet, Paxil oral |
| | Phenelzine 15 mg tablet |
| | Pristiq 25, 50, 100 mg tablet, extended release, Pristiq oral |
| | Protriptyline 5 mg tablet |
| | Prozac 20, 40 mg capsule, Prozac oral |
| | Remeron oral |
| | Savella 25, 50, 100 mg tablet, Savella oral |
| | Savella 12.5 mg (5)-25 mg(8)-50 mg(42) tablets in a dose pack |
| | Sertraline 25, 50, 100 mg tablet, Sertraline oral |
| | Sertraline 20 mg/ml oral concentrate |
| | Silenor 3, 6 mg tablet, Silenor oral |
| | St. John's wort 300 mg capsule, St. John's wort oral |
| | Trazodone 50, 100, 150, 300 mg tablet, Trazodone oral |
| | Trazodone ER 150 mg tablet, extended release 24 hr |
| | Trintellix 5, 10, 20 mg tablet, Trintellix oral |
| | Venlafaxine 25, 37.5, 50, 75, 100 mg tablet, Venlafaxine oral |
| | Venlafaxine ER 37.5, 75, 150 mg capsule, extended release 24 hr |
| | Venlafaxine ER 37.5, 75, 150, 225 mg tablet, extended release 24 hr |
| | Viibryd 10, 20, 40 mg tablet |
| | Vilazodone 10 mg (7)-20 mg (23) tablets in a titration pack |
| | Vilazodone 10 mg (7)-20 mg (7)-40 mg(16) tablets in a titration pack |
| | Vilazodone 10, 20, 40 mg tablet |
| | Vortioxetine 5, 10, 20 mg tablet |
| | Wellbutrin 100 mg tablet, Wellbutrin oral |
| | Wellbutrin XL oral |
| | Zoloft 100 mg tablet, Zoloft oral |
| Aspirin Medication | Aggrenox oral |
| | Aspir-81 mg tablet, delayed release, Aspir-81 oral |
| | Aspir-low 81 mg tablet, delayed release |
| | Aspirin (bulk) 100% powder |
| | Aspirin 25 mg-dipyridamole 200 mg capsule, ext. release 12 hr multiphase |
| | Aspirin 325, 500 mg tablet, Aspirin oral |
| | Aspirin 325, 650 mg tablet, delayed release |
| | Aspirin 81 mg chewable tablet |
| | Aspirin 81 mg tablet |
| | Aspirin 81 mg tablet, delayed release |
| | Aspirin low dose oral |
| | Aspirin-caffeine 400 mg-32 mg, 500 mg-32.5 mg tablet |
| | Aspirin-calcium carbonate 81 mg-300 mg calcium (777 mg) tablet |
| | Aspirin, buffered 325, 500 mg tablet |
| | Baby aspirin oral |
| | Back and body pain reliever oral |
| | Bayer aspirin oral |
| | Bayer back and body oral |
| | Bayer childrens aspirin oral |
| | Ecotrin low strength 81 mg tablet, enteric coated |
| | Ecotrin maximum strength oral |
| | Fiorinal oral |
| Celecoxib Medication | Celebrex 100 mg capsule, Celebrex oral |
| | Celecoxib 50, 100, 200, 400 mg capsule |
| Meloxicam Medication | Meloxicam 7.5, 15 mg tablet, Meloxicam oral |
| | Meloxicam 7.5 mg disintegrating tablet |
| | Meloxicam 7.5 mg/5 ml oral suspension |
| | Mobic 15 mg tablet, Mobic oral |
| Muscle Relaxant Medications | Abobotulinumtoxina 500 unit intramuscular solution |
| | Baclofen (bulk) 100% powder |
| | Baclofen 5, 10, 20 mg tablet, Baclofen oral |
| | Baclofen 10 mg/gabapentin 150 mg vaginal suppository |
| | Baclofen 10,000 mcg/20 ml (500 mcg/ml), 40,000 mcg/20 ml (2,000 mcg/ml) intrathecal solution |
| | Baclofen 10,000 mcg/20 ml (500 mcg/ml), 40,000 mcg/20 ml (2,000 mcg/ml) intrathecal syringe |
| | Baclofen 10 mg/1 ml |
| | Baclofen 50,500, 2,000 mcg/ml intrathecal solution |
| | Baclofen 7.5 mg elixir |
| | Baclofen intrathecal injection, Baclofen it |
| | Botox 100, 200 unit injection, Botox inj |
| | Carisoprodol 250, 350 mg tablet, Carisoprodol oral |
| | Chlorzoxazone 250, 500 mg tablet, Chlorzoxazone oral |
| | Cyclobenzaprine (bulk) 100% powder |
| | Cyclobenzaprine 5, 7.5, 10 mg tablet, Cyclobenzaprine oral |
| | Cyclobenzaprine ER15 mg capsule, extended release 24 hr |
| | Dantrolene 25, 50, 100 mg capsule |
| | Dysport 300, 500 unit intramuscular solution |
| | Flexeril oral |
| | Gablofen 40,000 mcg/20 ml (2,000 mcg/ml) intrathecal solution |
| | Gablofen 40,000 mcg/20 ml (2,000 mcg/ml) intrathecal syringe |
| | Lioresal 500, 2,000 mcg/ml intrathecal solution |
| | Lorzone 750 mg tablet |
| | Metaxalone 400, 800 mg tablet, Metaxalone oral |
| | Methocarbamol 500, 750 mg tablet, Methocarbamol oral |
| | Norflex oral |
| | Onabotulinumtoxina (botox) 10 units/0.1 ml (1:1) injection |
| | Onabotulinumtoxina 100, 200 unit solution for injection |
| | Orphenadrine citrate ER 100 mg tablet, extended release |
| | Orphenadrine citrate oral |
| | Parafon forte DSC oral |
| | Robaxin 500 mg tablet, Robaxin-750 oral, Robaxin oral |
| | Skelaxin 800 mg tablet, Skelaxin oral |
| | Soma oral |
| | Tizanidine 2, 4, 6 mg capsule, Tizanidine 2, 4 mg tablet, Tizanidine oral |
| | Zanaflex 4 mg capsule, Zanaflex oral |
| Naproxen Medication | Aleve 220 mg capsule, Aleve 220 mg tablet, Aleve oral |
| | Aleve cold and sinus oral |
| | Aleve pm oral |
| | Naprosyn oral |
| | Naproxen 125 mg/5 ml oral suspension |
| | Naproxen 220 mg-diphenhydramine 25 mg tablet |
| | Naproxen 250, 375, 500 mg tablet, Naproxen oral |
| | Naproxen 375, 500 mg tablet, delayed release |
| | Naproxen 500 mg-esomeprazole 20 mg tablet, immediate and delay release |
| | Naproxen sodium 220 mg capsule, Naproxen sodium 220, 275, 550 mg tablet, Naproxen sodium oral |
| | Naproxen sodium ER (CR) 375, 500, 750 mg tablet, extended release 24 hr mphase |
| Schedule III/IV Opioid Medications | Acetaminophen 120 mg-codeine 12 mg/5 ml oral solution |
| | Acetaminophen 300 mg-codeine 15, 30, 60 mg tablet, Acetaminophen-codeine oral |
| | Ascomp with codeine 30 mg-50 mg-325 mg-40 mg capsule |
| | Belbuca 75, 150, 450, 750 mcg buccal film, Belbucabucl |
| | Buprenorphine 0.7 mg-naloxone 0.18 mg sublingual tablet |
| | Buprenorphine 5.7 mg-naloxone 1.4 mg sublingual tablet |
| | Buprenorphine 8 mg-naloxone 2 mg sublingual tablet |
| | Buprenorphine 8.6 mg-naloxone 2.1 mg sublingual tablet |
| | Buprenorphine 12 mg-naloxone 3 mg sublingual film |
| | Buprenorphine 2 mg-naloxone 0.5 mg sublingual film |
| | Buprenorphine 4 mg-naloxone 1 mg sublingual film |
| | Buprenorphine 8 mg-naloxone 2 mg sublingual film |
| | Buprenorphine 5, 7.5, 10, 15, 20 mcg/hour weekly transdermal patch |
| | Buprenorphine Hcl 75, 150, 300, 450, 600 mcg buccal film |
| | Buprenorphine Hcl 8 mg sublingual tablet, Buprenorphine Hcl sl |
| | Buprenorphine-naloxone sl |
| | Butalbital 25 mg-acetaminophen 325 mg tablet |
| | Butalbital 50 mg-acetaminophen 300, 325 mg-caffeine 40 mg-codeine 30 mg cap |
| | Butalbital compound with codeine 30 mg-50 mg-325 mg-40 mg capsule |
| | Butalbital-acetaminophen 50 mg-325 mg tablet |
| | Butalbital-acetaminophen-caff oral |
| | Butalbital-acetaminophen-caffeine 50 mg-300, 325 mg-40 mg capsule |
| | Butalbital-acetaminophen-caffeine 50 mg-325, 500 mg-40 mg tablet |
| | Butalbital-aspirin-caffeine 50 mg-325 mg-40 mg capsule |
| | Butalbital-aspirin-caffeine 50 mg-325 mg-40 mg tablet |
| | Butorphanol tartrate 10 mg/ml nasal spray |
| | Butrans 5, 7.5, 10, 15, 20 mcg/hour transdermal patch |
| | Codeine 10 mg-guaifenesin 100 mg/5 ml oral liquid |
| | Codeine sulfate 15, 30, 60 mg tablet, Codeine sulfate oral |
| | Codeine-butalbital-asa-caffeine 30 mg-50 mg-325 mg-40 mg capsule |
| | Eluxadoline 100 mg tablet |
| | Fioricet 50 mg-300 mg-40 mg capsule, Fioricet oral |
| | Guaiatussin ac 10 mg-100 mg/5 ml oral liquid |
| | lophen c-nr 10 mg-100 mg/5 ml oral liquid |
| | Paregoric 2 mg/5 ml oral liquid |
| | Pentazocine 50 mg-naloxone 0.5 mg tablet |
| | Promethazine 6.25 mg-codeine 10 mg/5 ml syrup |
| | Promethazine vc-codeine 6.25 mg-5 mg-10 mg/5 ml oral syrup |
| | Pseudoephedrine-codeine-gg 30 mg-10 mg-100 mg/5 ml oral syrup |
| | Suboxone 2 mg-0.5 mg, 4 mg-1 mg, 8 mg-2 mg, 12 mg-3 mg sublingual film, Suboxone sl |
| | Tramadol 37.5 mg-acetaminophen 325 mg tablet |
| | Tramadol 50 mg disintegrating tablet |
| | Tramadol 50 mg tablet |
| | Tramadol ER 100 mg (ultram er) 24 hr tablet |
| | Tramadol ER100, 150, 200 mg capsule 24 h, extended release(25-75) |
| | Tramadol ER 100, 200 mg tablet, extended release 24 hr mphase |
| | Tramadol ER 200, 300 mg tablet, extended release 24 hr |
| | Tramadol ER 300 mg capsule 24 hr, extended release |
| | Tramadol oral |
| | Tramadol-acetaminophen oral |
| | Tylenol-codeine #3, #4 oral |
| | Ultram oral |
| | Zubsolv 2.9 mg-0.71 mg, 5.7 mg-1.4 mg sublingual tablet |
| Abdominal and Pelvic Blocks | Ganglion impar injection |
| | Inj anes ilio nerve lt, rt |
| | Inj anesth pudendal nerve |
| | Inj celiac plexus lt, rt |
| | Inject nerv blck, celiac plexus, |
| | Inject nerv blck, ilioingu/iliohyp |
| | Injection, anesthetic agent ilionguinal, iliohypogastric nerves |
| | N block inj, hypogas plxs |
| | Tap block bi injection |
| | Tap block single unilateral lt, rt |
| | Tap block unil by injection |
| Cervical Epidural Injections | Inj foramen epidural c/t |
| | Inj transfrmn cer/thor sin lt, rt |
| | Njx dx/ther sbst intrlmnr crv/thrc w/img gdn |
| | Njx dx/ther sbst intrlmnr crv/thrc w/o img gdn |
| Interlaminar Lumbar Epidural Injections | Njx dx/ther sbst intrlmnr lmbr/sac w/img gdn |
| | Njx dx/ther sbst intrlmnr lmbr/sac w/o img gdn |
| Muscle and Tendons Injection | Inj sg/mul trg pts 1-2 muscles |
| | Inj sn/mul tg pt 1-2 mus gr lt, rt |
| | Inj tendon/ligament/cyst |
| | Inj trigger point =>3 mgrps lt, rt |
| | Inject tendon origin/insert |
| | Inject trigger points, >3 |
| Pain Pumps and Other Implanted Devices | Analyze infusn pump + reprogram |
| | Anl sp inf pmp w/mdreprg&fil |
| | Complex neurostim analyze, 1st hour |
| | Electrical stimulator supplies, 2 lead, per month (e.g., tens, nmes) |
| | Percut implnt neuroelect, epidural |
| | Spin/brain pump refil & main |
| Behavioral Medicine | Assess hlth/behave, init |
| | Behav chng smoking 3-10 min, >10 min |
| | Consult/referral to alcohol & drug treatment |
| | Consult/referral to behavioral health program |
| | Consult/referral to neuropsychology |
| | Consult/referral to neuropsychology clinic |
| | Consult/referral to opioid treatment center |
| | Consult/referral to psychiatry |
| | Consult/referral to psychology |
| | Consult/referral to psychology evaluation |
| | Consult/referral to social work |
| | Consult/referral to supportive care clinic |
| | Consult/referral to treatment resistant depression (optimum) |
| | Depression screen positive w follow-up |
| | Enrollment in community services |
| | Group health education |
| | Individual psychotherapy |
| | Intervene hlth/behave, group, indiv |
| | Neuropsych testing adult |
| | Psychotherapy e/m 20-30, 45-50 mins |
| | Psychotherapy pt&/family 30 minutes |
| | Psychotherapy w/e&m 30 min |
| | Smoking cessation counseling |
| | Social work visit, in the home, Social worker visit |
| | Social worker nc |
| | Tobacco cessation counseling. Tobacco counseling |
| Integrative Medicine | Acupunct w/o stimul 15 min |
| | Acupunct w/o stimul addl 15 m |
| | Acupunct w/stimul 15 min |
| | Acupunct w/stimul addl 15 m |
| | Acupuncture w electrical stim |
| | Acupuncture w stim 1st 15 mn sp |
| | Acupuncture w/o electrical stim |
| | Acupuncture; extended |
| | Consult/referral for acupuncture |
| | Consult/referral to chiropractic therapy |
| | Consult/referral to complementary medicine department |
| | Consult/referral to massage therapy |
| | Consult/referral to osteopathic manipulative therapy |
| | Consult/referral to upmc center integrative medicine |
| | Massage therapy |
| | Yoga class |
| Rehabilitation Therapies | Aqua w/therapeutic excercise-each 15 min |
| | Aquatic therapy/exercises |
| | Consult/referral to aqua therapy |
| | Consult/referral to aquatic program |
| | Consult/referral to balance training |
| | Consult/referral to center for sports medicine |
| | Consult/referral to functional capacity evaluation |
| | Consult/referral to gait training |
| | Consult/referral to hand therapy |
| | Consult/referral to kinesthetic sense training |
| | Consult/referral to occupational therapy |
| | Consult/referral to occupational therapy for continuing care |
| | Consult/referral to occupational therapy for treatment |
| | Consult/referral to pelvic floor physiotherapy |
| | Consult/referral to physical medicine and rehabilitation |
| | Consult/referral to physical therapy |
| | Consult/referral to physical therapy for continuing care |
| | Consult/referral to vocational rehabilitation program |
| | Consult/referral to womens rehabilitation |
| | Consult/referral to work hardening program |
| | Home exercise program |
| | Hydrotherapy |
| | Occupational therapy eval, Occupational therapy evaluation |
| | OT functional assessment |
| | Physical therapy evaluation |
| | Physical therapy treatment |
| Orthotics and Assistive Devices | Addn to ctlso thoracic pad |
| | Afo ankle gauntlet, custom fitted |
| | Afo custom fitted, plastic |
| | Air pressure pad/cushion |
| | Ancillary orthotic services |
| | Apply finger splint, static |
| | Bath/shower chair (UPMC DME) |
| | Bedside commode (UPMC DME) |
| | Cane (UPMC DME), Cane, quad or three prong |
| | Cervical traction equipment |
| | Cervical, flexible, non-adjustable |
| | Collar cervical medium |
| | Consult/referral to center for assistive technology |
| | Consult/referral to durable medical equipment |
| | Consult/referral to orthotic clinic |
| | Consult/referral to prosthetic clinic |
| | Crutches underarm other than wood adjustable or fixed pair |
| | Crutches, forearm |
| | Crutches, underarm aluminum |
| | Custom orthotics |
| | DME supply or accessory, nos |
| | Elastic supp/stock bk med weight |
| | Finger splint static |
| | Folding walker, wheeled |
| | G compression stocking |
| | Gloves |
| | Heavy duty wheeled walker |
| | HFO, no joint, prefabricated |
| | Hospital bed (UPMC DME) |
| | Infrared heating pad system |
| | Ko elastic w/condylar pads/joints |
| | Ko elastic w/joints |
| | Lo flexibl l1-below l5 pre |
| | LSO custom fit |
| | LSO flexible, elastic |
| | LSO lumbar flexion |
| | LSO sag-coro rigid frame pre |
| | LSO sagit rigid panel prefab |
| | LSO, full corset |
| | Lumbar orthosis/brace (UPMC DME) |
| | Misc durable medical equip |
| | Ortho shoe custom shoes |
| | Orthotics fitting/tmg-each 15 min |
| | Ot splint hfo static finger fl/ex |
| | Other accessory |
| | Patient lift, electric |
| | Power air mattress overlay |
| | Raised toilet seat |
| | Repair of orthotic device |
| | Repl tip cane/crutch/walker |
| | Resting hand splint |
| | Sacroiliac flexible custom fabr |
| | Semi-rigid adj cerv molded chin cup |
| | Shoe lift |
| | Sling, arm small |
| | Splint |
| | Splint wrist or ankle |
| | Standard wheelchair |
| | TLSO flex prefab |
| | TLSO flexible custom fitted |
| | Transfer tub rail attachment |
| | Transport chair, adult size |
| | Tub stool or bench |
| | Walker (UPMC DME) |
| | Walker, folding (pickup) |
| | Walker, rigid (pickup) |
| | Walker, wheeled, no seat, Walker, wheeled, seat, Walker wheeled with seat |
| | Wheelchair, Wheelchair (UPMC DME) |
| | WHFO wrist extension control |
| | WHFO wrist gauntlet w/thumb spica |
| | Wrist splint left |
| | Wrist splint right |

TABLE 7
Continuous variables showing the highest predictive validity in the random forest
models trained for the 19 chronic pain treatment categories and subcategories.

| | | Feature | Non-responders | | Responders | | |
|---|---|---|---|---|---|---|---|
| Dataset | Baseline Feature | Score | Mean (SD) | | Mean (SD) | | p |
| Abdominal and Pelvic Blocks | Sleep Disturbance T-Score | 0.0594 | 58.33 | (8.26) | 59.81 | (11.74) | 0.442 |
| Abdominal and Pelvic Blocks | Neuropathic Pain Score | 0.0558 | 15.40 | (8.42) | 13.65 | (8.97) | 0.301 |
| Abdominal and Pelvic Blocks | Pain Interference T-Score | 0.0447 | 66.52 | (6.61) | 65.41 | (7.43) | 0.415 |
| Abdominal and Pelvic Blocks | Global Mental Health T-Score | 0.0443 | 40.39 | (8.42) | 41.98 | (9.18) | 0.351 |
| Abdominal and Pelvic Blocks | Number of Body Map Regions | 0.0393 | 7.86 | (11.27) | 5.60 | (7.46) | 0.242 |
| Abdominal and Pelvic Blocks | Age | 0.0389 | 49.02 | (15.77) | 51.78 | (14.42) | 0.353 |
| Abdominal and Pelvic Blocks | Depression T-Score | 0.0383 | 55.79 | (10.91) | 56.75 | (11.23) | 0.656 |
| Abdominal and Pelvic Blocks | Global Physical Health T-Score | 0.0347 | 33.76 | (6.04) | 35.05 | (7.65) | 0.330 |
| Abdominal and Pelvic Blocks | Pain Behavior T-Score | 0.0333 | 61.87 | (2.00) | 61.73 | (3.04) | 0.774 |
| Abdominal and Pelvic Blocks | Anxiety T-Score | 0.0291 | 56.37 | (10.48) | 57.67 | (11.26) | 0.537 |
| Anticonvulsant Medications | Neuropathic Pain Score | 0.0423 | 17.00 | (8.39) | 15.07 | (8.35) | <0.001 |
| Anticonvulsant Medications | Global Mental Health T-Score | 0.0414 | 42.57 | (7.77) | 44.66 | (7.93) | <0.001 |
| Anticonvulsant Medications | Anxiety T-Score | 0.0375 | 57.12 | (9.89) | 55.11 | (10.00) | <0.001 |
| Anticonvulsant Medications | Sleep Disturbance T-Score | 0.0368 | 60.37 | (8.98) | 58.47 | (9.33) | <0.001 |
| Anticonvulsant Medications | Depression T-Score | 0.0362 | 55.99 | (10.30) | 53.98 | (10.35) | <0.001 |
| Anticonvulsant Medications | Age | 0.0337 | 56.07 | (14.43) | 57.70 | (14.32) | 0.003 |
| Anticonvulsant Medications | Pain Interference T-Score | 0.0337 | 66.53 | (5.94) | 65.96 | (6.46) | 0.014 |
| Anticonvulsant Medications | Number of Body Map Regions | 0.0328 | 12.15 | (11.02) | 10.13 | (9.93) | <0.001 |
| Anticonvulsant Medications | Pain Behavior T-Score | 0.0292 | 61.19 | (2.59) | 60.81 | (2.98) | <0.001 |
| Anticonvulsant Medications | Global Physical Health T-Score | 0.0268 | 33.47 | (5.85) | 34.39 | (6.74) | <0.001 |
| Anticonvulsant Medications | Pain Experience Duration | 0.0214 | 6.98 | (3.56) | 6.93 | (4.60) | 0.746 |
| Antidepressant Medications | Neuropathic Pain Score | 0.0473 | 18.14 | (8.20) | 15.70 | (8.75) | <0.001 |
| Antidepressant Medications | Anxiety T-Score | 0.0435 | 57.99 | (10.16) | 57.85 | (10.61) | 0.787 |
| Antidepressant Medications | Sleep Disturbance T-Score | 0.0430 | 61.20 | (8.76) | 60.34 | (9.71) | 0.067 |
| Antidepressant Medications | Age | 0.0425 | 53.55 | (13.27) | 55.48 | (13.93) | 0.006 |
| Antidepressant Medications | Depression T-Score | 0.0402 | 57.11 | (10.32) | 56.55 | (10.89) | 0.306 |
| Antidepressant Medications | Pain Interference T-Score | 0.0376 | 66.90 | (5.62) | 66.52 | (6.68) | 0.212 |
| Antidepressant Medications | Pain Behavior T-Score | 0.0298 | 61.60 | (2.14) | 61.35 | (2.38) | 0.031 |
| Antidepressant Medications | Number of Body Map Regions | 0.0294 | 14.01 | (12.92) | 11.61 | (11.98) | <0.001 |
| Antidepressant Medications | Global Mental Health T-Score | 0.0267 | 40.57 | (7.56) | 41.51 | (8.20) | 0.020 |
| Antidepressant Medications | Global Physical Health T-Score | 0.0258 | 32.49 | (5.28) | 33.50 | (6.40) | 0.001 |
| Aspirin NSAID Medication | Anxiety T-Score | 0.0532 | 56.06 | (9.30) | 52.76 | (10.83) | 0.035 |
| Aspirin NSAID Medication | Sleep Disturbance T-Score | 0.0426 | 58.83 | (10.03) | 55.59 | (10.13) | 0.043 |
| Aspirin NSAID Medication | Depression T-Score | 0.0371 | 54.97 | (10.20) | 51.49 | (10.38) | 0.033 |
| Aspirin NSAID Medication | Pain Interference T-Score | 0.0352 | 66.20 | (5.97) | 65.74 | (5.90) | 0.620 |
| Aspirin NSAID Medication | Number of Body Map Regions | 0.0336 | 9.94 | (9.02) | 11.74 | (13.09) | 0.290 |
| Aspirin NSAID Medication | Global Mental Health T-Score | 0.0320 | 41.79 | (7.74) | 45.86 | (9.25) | 0.002 |
| Aspirin NSAID Medication | Age | 0.0318 | 62.88 | (12.68) | 63.14 | (14.02) | 0.900 |
| Aspirin NSAID Medication | Global Physical Health T-Score | 0.0318 | 33.05 | (6.08) | 34.40 | (6.09) | 0.161 |
| Aspirin NSAID Medication | Neuropathic Pain Score | 0.0286 | 16.20 | (8.54) | 13.57 | (7.88) | 0.046 |
| Aspirin NSAID Medication | Pain Behavior T-Score | 0.0285 | 60.42 | (2.99) | 60.51 | (3.03) | 0.841 |
| Aspirin NSAID Medication | Positive Outlook T-Score | 0.0223 | 49.28 | (3.62) | 50.27 | (5.37) | 0.152 |
| Behavioral Medicine | Sleep Disturbance T-Score | 0.0413 | 62.89 | (8.41) | 64.31 | (8.82) | 0.182 |
| Behavioral Medicine | Depression T-Score | 0.0396 | 61.39 | (8.21) | 62.49 | (9.73) | 0.325 |
| Behavioral Medicine | Neuropathic Pain Score | 0.0348 | 18.86 | (8.69) | 17.68 | (9.47) | 0.293 |
| Behavioral Medicine | Global Mental Health T-Score | 0.0341 | 37.97 | (5.92) | 37.32 | (7.92) | 0.462 |
| Behavioral Medicine | Anxiety T-Score | 0.0323 | 62.15 | (9.13) | 62.90 | (8.32) | 0.475 |
| Behavioral Medicine | Age | 0.0276 | 49.89 | (12.91) | 48.92 | (13.45) | 0.547 |
| Behavioral Medicine | Pain Interference T-Score | 0.0269 | 68.05 | (6.02) | 69.35 | (5.01) | 0.052 |
| Behavioral Medicine | Pain Behavior T-Score | 0.0250 | 62.42 | (2.17) | 62.49 | (1.91) | 0.795 |
| Behavioral Medicine | Number of Body Map Regions | 0.0234 | 20.41 | (15.38) | 20.18 | (16.31) | 0.905 |
| Celecoxib NSAID Medication | Pain Interference T-Score | 0.0487 | 65.94 | (5.68) | 66.22 | (6.65) | 0.649 |
| Celecoxib NSAID Medication | Depression T-Score | 0.0481 | 55.92 | (10.04) | 54.08 | (11.37) | 0.094 |
| Celecoxib NSAID Medication | Anxiety T-Score | 0.0470 | 56.88 | (9.53) | 54.48 | (10.06) | 0.018 |
| Celecoxib NSAID Medication | Sleep Disturbance T-Score | 0.0375 | 59.75 | (8.57) | 58.52 | (10.06) | 0.194 |
| Celecoxib NSAID Medication | Number of Body Map Regions | 0.0346 | 12.08 | (10.97) | 9.39 | (10.42) | 0.017 |
| Celecoxib NSAID Medication | Age | 0.0339 | 58.47 | (13.19) | 62.03 | (12.41) | 0.008 |
| Celecoxib NSAID Medication | Pain Behavior T-Score | 0.0311 | 60.63 | (2.73) | 60.74 | (2.29) | 0.693 |
| Celecoxib NSAID Medication | Neuropathic Pain Score | 0.0309 | 16.89 | (8.22) | 14.77 | (7.98) | 0.012 |
| Celecoxib NSAID Medication | Global Physical Health T-Score | 0.0271 | 33.35 | (5.73) | 33.44 | (6.33) | 0.892 |
| Celecoxib NSAID Medication | Global Mental Health T-Score | 0.0269 | 43.39 | (7.64) | 45.16 | (8.03) | 0.029 |
| Celecoxib NSAID Medication | Positive Outlook T-Score | 0.0161 | 50.35 | (4.68) | 51.81 | (5.16) | 0.004 |
| Cervical Epidural Injections | Neuropathic Pain Score | 0.0444 | 15.81 | (7.90) | 13.25 | (8.59) | 0.008 |
| Cervical Epidural Injections | Anxiety T-Score | 0.0374 | 55.27 | (10.15) | 53.27 | (10.30) | 0.094 |
| Cervical Epidural Injections | Sleep Disturbance T-Score | 0.0366 | 60.36 | (8.41) | 58.60 | (9.05) | 0.083 |
| Cervical Epidural Injections | Age | 0.0342 | 57.10 | (12.63) | 56.24 | (12.59) | 0.560 |
| Cervical Epidural Injections | Pain Interference T-Score | 0.0307 | 64.21 | (6.51) | 63.94 | (6.71) | 0.725 |
| Cervical Epidural Injections | Global Physical Health T-Score | 0.0282 | 35.76 | (6.82) | 35.00 | (5.97) | 0.314 |
| Cervical Epidural Injections | Pain Behavior T-Score | 0.0282 | 59.92 | (2.52) | 59.71 | (3.24) | 0.529 |
| Cervical Epidural Injections | Depression T-Score | 0.0281 | 53.80 | (10.66) | 51.87 | (10.57) | 0.121 |
| Cervical Epidural Injections | Global Mental Health T-Score | 0.0256 | 44.63 | (7.68) | 44.89 | (7.94) | 0.771 |
| Cervical Epidural Injections | Number of Body Map Regions | 0.0254 | 11.08 | (10.52) | 9.64 | (8.42) | 0.203 |
| Integrative Medicine | Age | 0.0517 | 53.63 | (14.71) | 58.39 | (15.21) | 0.040 |
| Integrative Medicine | Sleep Disturbance T-Score | 0.0462 | 58.84 | (8.83) | 56.86 | (7.66) | 0.126 |
| Integrative Medicine | Number of Body Map Regions | 0.0437 | 14.90 | (12.37) | 12.48 | (14.72) | 0.244 |
| Integrative Medicine | Neuropathic Pain Score | 0.0323 | 14.93 | (7.94) | 13.26 | (7.15) | 0.157 |
| Integrative Medicine | Depression T-Score | 0.0284 | 54.24 | (10.36) | 52.22 | (8.35) | 0.171 |
| Integrative Medicine | Pain Interference T-Score | 0.0253 | 64.93 | (6.51) | 64.39 | (7.05) | 0.603 |
| Integrative Medicine | Positive Outlook T-Score | 0.0245 | 47.76 | (7.02) | 49.33 | (4.55) | 0.095 |
| Integrative Medicine | Global Physical Health T-Score | 0.0236 | 32.70 | (5.75) | 33.67 | (6.41) | 0.302 |
| Integrative Medicine | Global Mental Health T-Score | 0.0233 | 44.85 | (7.65) | 44.63 | (7.10) | 0.851 |
| Integrative Medicine | Anxiety T-Score | 0.0226 | 55.63 | (9.99) | 54.18 | (8.60) | 0.317 |
| Intralaminar Lumbar Epidural Injections | Pain Interference T-Score | 0.0402 | 65.26 | (5.80) | 65.33 | (5.98) | 0.811 |
| Intralaminar Lumbar Epidural Injections | Sleep Disturbance T-Score | 0.0396 | 57.71 | (8.99) | 56.72 | (8.70) | 0.019 |
| Intralaminar Lumbar Epidural Injections | Anxiety T-Score | 0.0395 | 53.68 | (10.25) | 52.94 | (9.44) | 0.117 |
| Intralaminar Lumbar Epidural Injections | Depression T-Score | 0.0374 | 52.85 | (10.47) | 51.79 | (9.62) | 0.028 |
| Intralaminar Lumbar Epidural Injections | Age | 0.037 | 61.86 | (14.41) | 62.74 | (13.87) | 0.191 |
| Intralaminar Lumbar Epidural Injections | Global Mental Health T-Score | 0.0367 | 45.03 | (7.52) | 47.07 | (7.23) | <0.001 |
| Intralaminar Lumbar Epidural Injections | Neuropathic Pain Score | 0.0359 | 14.11 | (7.88) | 13.51 | (7.40) | 0.099 |
| Intralaminar Lumbar Epidural Injections | Pain Behavior T-Score | 0.0338 | 60.13 | (2.52) | 59.92 | (2.88) | 0.101 |
| Intralaminar Lumbar Epidural Injections | Number of Body Map Regions | 0.0322 | 9.28 | (8.66) | 7.81 | (7.29) | <0.001 |
| Intralaminar Lumbar Epidural Injections | Global Physical Health T-Score | 0.0251 | 33.88 | (6.26) | 33.87 | (6.05) | 0.964 |
| Intralaminar Lumbar Epidural Injections | Positive Outlook T-Score | 0.0154 | 50.44 | (4.79) | 50.90 | (4.58) | 0.037 |
| Intralaminar Lumbar Epidural Injections | Pain Experience Duration | 0.0131 | 5.66 | (3.37) | 5.58 | (3.55) | 0.641 |
| Medications - Any Type Below | Neuropathic Pain Score | 0.0521 | 16.24 | (8.37) | 14.14 | (8.17) | <0.001 |
| Medications - Any Type Below | Depression T-Score | 0.039 | 55.80 | (10.17) | 53.99 | (10.35) | <0.001 |
| Medications - Any Type Below | Sleep Disturbance T-Score | 0.0379 | 60.21 | (8.76) | 58.69 | (9.37) | <0.001 |
| Medications - Any Type Below | Anxiety T-Score | 0.0374 | 56.55 | (9.77) | 55.08 | (9.89) | <0.001 |
| Medications - Any Type Below | Global Mental Health T-Score | 0.0358 | 42.86 | (7.60) | 44.69 | (8.23) | <0.001 |
| Medications - Any Type Below | Pain Interference T-Score | 0.0343 | 66.41 | (5.92) | 66.19 | (6.39) | 0.214 |
| Medications - Any Type Below | Pain Behavior T-Score | 0.0336 | 61.12 | (2.64) | 60.79 | (2.80) | <0.001 |
| Medications - Any Type Below | Age | 0.0333 | 56.36 | (14.78) | 57.80 | (14.65) | 0.001 |
| Medications - Any Type Below | Number of Body Map Regions | 0.0294 | 11.87 | (11.26) | 9.78 | (9.81) | <0.001 |
| Medications - Any Type Below | Global Physical Health T-Score | 0.0238 | 33.47 | (5.88) | 34.31 | (6.68) | <0.001 |
| Medications - Any Type Below | Pain Experience Duration | 0.0216 | 7.08 | (3.99) | 6.82 | (4.20) | 0.027 |
| Meloxicam NSAID Medication | Sleep Disturbance T-Score | 0.0459 | 60.47 | (8.83) | 58.32 | (10.33) | 0.003 |
| Meloxicam NSAID Medication | Neuropathic Pain Score | 0.0404 | 16.61 | (8.37) | 13.64 | (8.08) | <0.001 |
| Meloxicam NSAID Medication | Age | 0.0339 | 52.92 | (12.72) | 52.74 | (14.26) | 0.858 |
| Meloxicam NSAID Medication | Global Mental Health T-Score | 0.0338 | 43.10 | (7.76) | 45.46 | (8.43) | <0.001 |
| Meloxicam NSAID Medication | Pain Interference T-Score | 0.0335 | 66.23 | (6.00) | 65.72 | (6.48) | 0.283 |
| Meloxicam NSAID Medication | Anxiety T-Score | 0.0305 | 56.28 | (10.17) | 55.50 | (9.93) | 0.314 |
| Meloxicam NSAID Medication | Depression T-Score | 0.0299 | 55.39 | (10.67) | 53.93 | (10.19) | 0.071 |
| Meloxicam NSAID Medication | Number of Body Map Regions | 0.0292 | 11.32 | (11.14) | 9.82 | (9.65) | 0.066 |
| Meloxicam NSAID Medication | Pain Behavior T-Score | 0.0282 | 61.01 | (2.52) | 60.67 | (2.92) | 0.104 |
| Meloxicam NSAID Medication | Global Physical Health T-Score | 0.0235 | 33.14 | (5.68) | 33.97 | (6.61) | 0.073 |
| Meloxicam NSAID Medication | Pain Experience Duration | 0.0126 | 5.80 | (3.49) | 5.46 | (2.90) | 0.181 |
| Multimodal Pain Management | Neuropathic Pain Score | 0.0476 | 15.60 | (8.53) | 13.65 | (8.13) | <0.001 |
| Multimodal Pain Management | Pain Behavior T-Score | 0.0392 | 60.98 | (2.99) | 60.66 | (3.04) | <0.001 |
| Multimodal Pain Management | Sleep Disturbance T-Score | 0.0343 | 60.21 | (8.76) | 58.69 | (9.37) | <0.001 |
| Multimodal Pain Management | Depression T-Score | 0.0317 | 55.64 | (10.24) | 53.87 | (10.38) | <0.001 |
| Multimodal Pain Management | Anxiety T-Score | 0.0316 | 56.38 | (9.84) | 54.91 | (9.88) | <0.001 |
| Multimodal Pain Management | Pain Interference T-Score | 0.0309 | 66.35 | (5.97) | 65.98 | (6.48) | 0.018 |
| Multimodal Pain Management | Global Mental Health T-Score | 0.0308 | 43.55 | (7.52) | 45.12 | (8.14) | <0.001 |
| Multimodal Pain Management | Age | 0.0304 | 56.81 | (15.14) | 58.31 | (15.18) | <0.001 |
| Multimodal Pain Management | Number of Body Map Regions | 0.0278 | 11.64 | (11.20) | 9.64 | (10.09) | <0.001 |
| Multimodal Pain Management | Global Physical Health T-Score | 0.0211 | 33.31 | (5.86) | 34.24 | (6.62) | <0.001 |
| Muscle and Tendon Injections | Sleep Disturbance T-Score | 0.0387 | 59.99 | (9.15) | 58.26 | (9.13) | 0.044 |
| Muscle and Tendon Injections | Age | 0.0381 | 55.92 | (16.30) | 58.13 | (14.62) | 0.130 |
| Muscle and Tendon Injections | Neuropathic Pain Score | 0.0373 | 14.93 | (8.54) | 13.70 | (7.16) | 0.099 |
| Muscle and Tendon Injections | Depression T-Score | 0.0372 | 54.62 | (10.73) | 52.30 | (10.57) | 0.020 |
| Muscle and Tendon Injections | Global Physical Health T-Score | 0.0362 | 33.84 | (5.00) | 35.22 | (7.49) | 0.017 |
| Muscle and Tendon Injections | Anxiety T-Score | 0.0356 | 55.87 | (10.68) | 54.20 | (10.15) | 0.088 |
| Muscle and Tendon Injections | Global Mental Health T-Score | 0.0342 | 42.41 | (8.06) | 44.84 | (8.28) | 0.002 |
| Muscle and Tendon Injections | Number of Body Map Regions | 0.0335 | 11.94 | (13.00) | 8.54 | (10.23) | 0.002 |
| Muscle and Tendon Injections | Pain Interference T-Score | 0.0332 | 64.97 | (5.97) | 64.67 | (6.54) | 0.613 |
| Muscle and Tendon Injections | Pain Behavior T-Score | 0.0245 | 60.48 | (2.36) | 60.26 | (2.38) | 0.337 |
| Muscle and Tendon Injections | Pain Experience Duration | 0.0084 | 3.93 | (1.94) | 3.68 | (1.49) | 0.139 |
| Muscle Relaxant Medications | Neuropathic Pain Score | 0.0467 | 17.52 | (8.23) | 15.30 | (8.56) | <0.001 |
| Muscle Relaxant Medications | Sleep Disturbance T-Score | 0.045 | 61.09 | (8.46) | 60.02 | (9.42) | 0.005 |
| Muscle Relaxant Medications | Pain Interference T-Score | 0.0421 | 66.82 | (5.59) | 66.97 | (6.50) | 0.557 |
| Muscle Relaxant Medications | Depression T-Score | 0.0418 | 56.32 | (10.07) | 54.75 | (10.63) | <0.001 |
| Muscle Relaxant Medications | Anxiety T-Score | 0.04 | 57.18 | (9.71) | 55.92 | (10.31) | 0.003 |
| Muscle Relaxant Medications | Age | 0.0391 | 53.66 | (13.43) | 54.55 | (13.34) | 0.125 |
| Muscle Relaxant Medications | Global Mental Health T-Score | 0.035 | 41.91 | (7.56) | 43.49 | (8.63) | <0.001 |
| Muscle Relaxant Medications | Pain Behavior T-Score | 0.0312 | 61.46 | (2.20) | 61.32 | (2.36) | 0.16 |
| Muscle Relaxant Medications | Number of Body Map Regions | 0.0307 | 13.27 | (12.57) | 11.42 | (11.25) | <0.001 |
| Muscle relaxant medications | Global Physical Health T-Score | 0.025 | 33.00 | (5.69) | 33.53 | (6.39) | 0.039 |
| Naproxen NSAID Medication | Depression T-Score | 0.052 | 56.28 | (10.56) | 53.42 | (10.35) | 0.046 |
| Naproxen NSAID Medication | Global Physical Health T-Score | 0.0424 | 32.54 | (5.72) | 35.06 | (7.13) | 0.004 |
| Naproxen NSAID Medication | Global Mental Health T-Score | 0.0416 | 42.62 | (7.41) | 46.70 | (7.63) | <0.001 |
| Naproxen NSAID Medication | Age | 0.0373 | 55.41 | (14.57) | 56.81 | (13.70) | 0.471 |
| Naproxen NSAID Medication | Anxiety T-Score | 0.0364 | 57.31 | (10.00) | 54.87 | (9.98) | 0.075 |
| Naproxen NSAID Medication | Neuropathic Pain Score | 0.0364 | 16.54 | (8.86) | 14.36 | (8.36) | 0.066 |
| Naproxen NSAID Medication | Pain Interference T-Score | 0.0323 | 66.25 | (5.87) | 65.93 | (6.27) | 0.703 |
| Naproxen NSAID Medication | Sleep Disturbance T-Score | 0.0298 | 60.64 | (8.15) | 59.35 | (8.72) | 0.260 |
| Naproxen NSAID Medication | Pain Behavior T-Score | 0.0261 | 61.20 | (2.51) | 60.67 | (2.86) | 0.147 |
| Naproxen NSAID Medication | Number of Body Map Regions | 0.023 | 10.82 | (10.59) | 9.94 | (11.17) | 0.553 |
| Orthotics and Assistive Devices | Sleep Disturbance T-Score | 0.065 | 60.68 | (7.92) | 57.66 | (9.29) | 0.052 |
| Orthotics and Assistive Devices | Anxiety T-Score | 0.0646 | 57.37 | (10.93) | 55.69 | (10.81) | 0.403 |
| Orthotics and Assistive Devices | Pain Behavior T-Score | 0.0548 | 61.64 | (1.85) | 61.29 | (1.84) | 0.290 |
| Orthotics and Assistive Devices | Global Physical Health T-Score | 0.0453 | 32.62 | (5.27) | 32.41 | (6.01) | 0.840 |
| Orthotics and Assistive Devices | Depression T-Score | 0.0416 | 55.97 | (10.80) | 54.96 | (10.70) | 0.611 |
| Orthotics and Assistive Devices | Global Mental Health T-Score | 0.0347 | 40.32 | (8.17) | 41.37 | (7.62) | 0.472 |
| Orthotics and Assistive Devices | Pain Interference T-Score | 0.0301 | 66.70 | (5.83) | 66.31 | (7.06) | 0.735 |
| Orthotics and Assistive Devices | Neuropathic Pain Score | 0.0273 | 17.13 | (7.83) | 15.46 | (8.57) | 0.262 |
| Orthotics and Assistive Devices | Age | 0.0258 | 59.65 | (15.85) | 59.76 | (15.74) | 0.970 |
| Orthotics and Assistive Devices | Number of Body Map Regions | 0.0251 | 13.55 | (12.79) | 11.38 | (10.67) | 0.331 |
| Pain Pumps and Other Implanted Devices | Anxiety T-Score | 0.0464 | 57.33 | (10.02) | 54.17 | (9.29) | 0.047 |
| Pain Pumps and Other Implanted Devices | Number of Body Map Regions | 0.0442 | 13.25 | (12.62) | 10.89 | (10.83) | 0.227 |
| Pain Pumps and Other Implanted Devices | Depression T-Score | 0.0437 | 56.38 | (10.26) | 53.64 | (9.81) | 0.094 |
| Pain Pumps and Other Implanted Devices | Global Physical Health T-Score | 0.0428 | 33.82 | (6.08) | 35.03 | (6.54) | 0.226 |
| Pain Pumps and Other Implanted Devices | Sleep Disturbance T-Score | 0.0383 | 60.55 | (7.93) | 59.28 | (8.84) | 0.338 |
| Pain Pumps and Other Implanted Devices | Pain Interference T-Score | 0.0371 | 66.92 | (5.43) | 66.08 | (5.72) | 0.341 |
| Pain Pumps and Other Implanted Devices | Global Mental Health T-Score | 0.0365 | 41.24 | (8.34) | 43.32 | (8.51) | 0.125 |
| Pain Pumps and Other Implanted Devices | Neuropathic Pain Score | 0.0318 | 17.71 | (7.69) | 16.56 | (8.38) | 0.369 |
| Pain Pumps and Other Implanted Devices | Age | 0.0307 | 54.38 | (13.88) | 57.17 | (12.93) | 0.205 |
| Pain Pumps and Other Implanted Devices | Pain Behavior T-Score | 0.0289 | 61.38 | (2.46) | 61.35 | (2.14) | 0.931 |
| Rehabilitation Therapies | Number of Body Map Regions | 0.0373 | 15.12 | (12.68) | 14.36 | (15.39) | 0.525 |
| Rehabilitation Therapies | Sleep Disturbance T-Score | 0.0372 | 60.47 | (8.81) | 59.07 | (9.94) | 0.083 |
| Rehabilitation Therapies | Age | 0.0358 | 55.04 | (13.83) | 55.50 | (16.24) | 0.721 |
| Rehabilitation Therapies | Pain Interference T-Score | 0.035 | 66.79 | (5.57) | 66.91 | (6.23) | 0.803 |
| Rehabilitation Therapies | Neuropathic Pain Score | 0.0344 | 16.68 | (8.49) | 15.57 | (9.17) | 0.144 |
| Rehabilitation Therapies | Depression T-Score | 0.0341 | 56.48 | (9.80) | 55.88 | (10.72) | 0.503 |
| Rehabilitation Therapies | Anxiety T-Score | 0.0322 | 57.36 | (9.66) | 56.66 | (9.89) | 0.410 |
| Rehabilitation Therapies | Pain Behavior T-Score | 0.0274 | 61.51 | (2.21) | 61.27 | (2.48) | 0.239 |
| Rehabilitation Therapies | Global Mental Health T-Score | 0.0222 | 42.18 | (7.22) | 43.45 | (7.66) | 0.050 |
| Rehabilitation Therapies | Global Physical Health T-Score | 0.0212 | 32.71 | (5.52) | 33.76 | (6.65) | 0.045 |
| Rehabilitation Therapies | Pain Experience Duration | 0.0144 | 7.93 | (5.16) | 6.74 | (3.14) | 0.003 |
| Schedule III/IV Opioid Medications | Neuropathic Pain Score | 0.0472 | 16.11 | (8.43) | 14.14 | (8.06) | <0.001 |
| Schedule III/IV Opioid Medications | Pain Interference T-Score | 0.0412 | 66.62 | (5.71) | 66.87 | (6.25) | 0.442 |
| Schedule III/IV Opioid Medications | Number of Body Map Regions | 0.0409 | 11.53 | (11.07) | 9.84 | (10.17) | 0.004 |
| Schedule III/IV Opioid Medications | Anxiety T-Score | 0.0393 | 56.33 | (9.95) | 55.19 | (9.75) | 0.034 |
| Schedule III/IV Opioid Medications | Sleep Disturbance T-Score | 0.0382 | 59.92 | (8.57) | 59.34 | (9.14) | 0.227 |
| Schedule III/IV Opioid Medications | Age | 0.0374 | 59.25 | (15.24) | 59.25 | (15.22) | 0.997 |
| Schedule III/IV Opioid Medications | Depression T-Score | 0.0358 | 55.59 | (10.18) | 54.66 | (9.96) | 0.092 |
| Schedule III/IV Opioid Medications | Global Mental Health T-Score | 0.0339 | 42.78 | (8.05) | 45.03 | (8.17) | <0.001 |
| Schedule III/IV Opioid Medications | Pain Behavior T-Score | 0.0306 | 61.22 | (2.43) | 61.15 | (2.36) | 0.612 |
| Schedule III/IV Opioid Medications | Global Physical Health T-Score | 0.0269 | 33.77 | (6.08) | 34.75 | (6.68) | 0.005 |

TABLE 8
Categorical variables showing the highest predictive validity in the random forest models trained for the 19 chronic pain treatments.

| | | Feature | % Non- | % | |
|---|---|---|---|---|---|
| Dataset | Baseline Feature | Score | responders | Responders | p |
| Abdominal and Pelvic Blocks | Pain in buttocks region | 0.0136 | 34.4 | 15.6 | 0.049 |
| Abdominal and Pelvic Blocks | Abdominal Pain cluster classification | 0.0304 | 28.1 | 57.8 | 0.038 |
| Anticonvulsant Medications | Opioid misuse behaviors unknown/not assessed | 0.0231 | 9.2 | 19.2 | <0.001 |
| Antidepressant Medications | Opioid misuse behaviors unknown/not assessed | 0.0176 | 7.4 | 18.3 | <0.001 |
| Antidepressant Medications | Prescription opioid user, few or no misuse behaviors | 0.0149 | 87.4 | 77.2 | <0.001 |
| Aspirin NSAID Medication | No Medicaid insurance | 0.0155 | 77.4 | 92.3 | 0.036 |
| Behavioral Medicine | No NSAID medication use | 0.0229 | 59.8 | 44.3 | 0.036 |
| Behavioral Medicine | No/unknown opioid use | 0.0270 | 19.7 | 35.4 | 0.006 |
| Behavioral Medicine | Prescription opioid user | 0.0253 | 80.3 | 64.6 | 0.006 |
| Celecoxib NSAID Medication | Pain in shoulder region | 0.0139 | 46.2 | 28.8 | 0.001 |
| Cervical Epidural Injections | Not currently on disability assistance | 0.0115 | 29.6 | 50.4 | 0.001 |
| Cervical Epidural Injections | No/unknown opioid use | 0.0109 | 26.6 | 36.6 | 0.083 |
| Integrative Medicine | Pain in lower back region | 0.0220 | 62.9 | 44 | 0.021 |
| Medications - Any Type Listed | Opioid misuse behaviors unknown/not assessed | 0.0322 | 8.7 | 19.8 | <0.001 |
| Multimodal Pain Management | Opioid misuse behaviors unknown/not assessed | 0.0412 | 5.3 | 195 | <0.001 |
| Multimodal Pain Management | Prescription opioid user, few or no misuse | 0.0318 | 88.8 | 76.3 | <0.001 |
| Muscle and Tendon Injections | Opioid misuse behaviors unknown/not assessed | 0.0105 | 8.3 | 22.4 | <0.001 |
| Muscle Relaxant Medications | Opioid misuse behaviors unknown/not assessed | 0.0269 | 7.9 | 20 | <0.001 |
| Muscle Relaxant Medications | Prescription opioid user, few or no misuse | 0.0146 | 84.9 | 74.9 | <0.001 |
| Naproxen NSAID Medication | Did not experience chronic pain as a child | 0.0181 | 38.5 | 56.6 | 0.033 |
| Naproxen NSAID Medication | Never hospitalized for psychiatric conditions | 0.0171 | 36.8 | 54.5 | 0.052 |
| Orthotics and Assistive Devices | Pain in hand region | 0.0200 | 20.5 | 28.9 | 0.383 |
| Pain Pumps and Other Implanted Devices | No pain in hip region | 0.0186 | 53.7 | 66.7 | 0.141 |
| Pain Pumps and Other Implanted Devices | Opioid misuse behaviors unknown/not assessed | 0.0199 | 9.6 | 25.9 | 0.014 |
| Schedule III/IV Opioid Medications | Pain in lower back region | 0.0246 | 64.1 | 49.4 | <0.001 |

### Computing and Processing Environment

The subject matter and the actions and operations described in this specification can be implemented in digital electronic circuitry, in tangibly-embodied computer software or firmware, in computer hardware, including the structures disclosed in this specification and their structural equivalents, or in combinations of one or more of them. The subject matter and the actions and operations described in this specification can be implemented as or in one or more computer programs, e.g., one or more modules of computer program instructions, encoded on a computer program carrier, for execution by, or to control the operation of, data processing apparatus. The carrier can be a tangible non-transitory computer storage medium. Alternatively or in addition, the carrier can be an artificially-generated propagated signal, e.g., a machine-generated electrical, optical, or electromagnetic signal, that is generated to encode information for transmission to suitable receiver apparatus for execution by a data processing apparatus. The computer storage medium can be or be part of a machine-readable storage device, a machine-readable storage substrate, a random or serial access memory device, or a combination of one or more of them. A computer storage medium is not a propagated signal.

The term “data processing apparatus” encompasses all kinds of apparatus, devices, and machines for processing data, including by way of example a programmable processor, a computer, or multiple processors or computers. Data processing apparatus can include special-purpose logic circuitry, e.g., an FPGA (field programmable gate array), an ASIC (application-specific integrated circuit), or a GPU (graphics processing unit). The apparatus can also include, in addition to hardware, code that creates an execution environment for computer programs, e.g., code that constitutes processor firmware, a protocol stack, a database management system, an operating system, or a combination of one or more of them.

A computer program can be written in any form of programming language, including compiled or interpreted languages, or declarative or procedural languages; and it can be deployed in any form, including as a stand-alone program, e.g., as an app, or as a module, component, engine, subroutine, or other unit suitable for executing in a computing environment, which environment may include one or more computers interconnected by a data communication network in one or more locations.

A computer program may, but need not, correspond to a file in a file system. A computer program can be stored in a portion of a file that holds other programs or data, e.g., one or more scripts stored in a markup language document, in a single file dedicated to the program in question, or in multiple coordinated files, e.g., files that store one or more modules, sub-programs, or portions of code.

The processes and logic flows described in this specification can be performed by one or more computers executing one or more computer programs to perform operations by operating on input data and generating output. The processes and logic flows can also be performed by special-purpose logic circuitry, e.g., an FPGA, an ASIC, or a GPU, or by a combination of special-purpose logic circuitry and one or more programmed computers.

Computers suitable for the execution of a computer program can be based on general or special-purpose microprocessors or both, or any other kind of central processing unit. Generally, a central processing unit will receive instructions and data from a read-only memory or a random access memory or both. The essential elements of a computer are a central processing unit for executing instructions and one or more memory devices for storing instructions and data. The central processing unit and the memory can be supplemented by, or incorporated in, special-purpose logic circuitry.

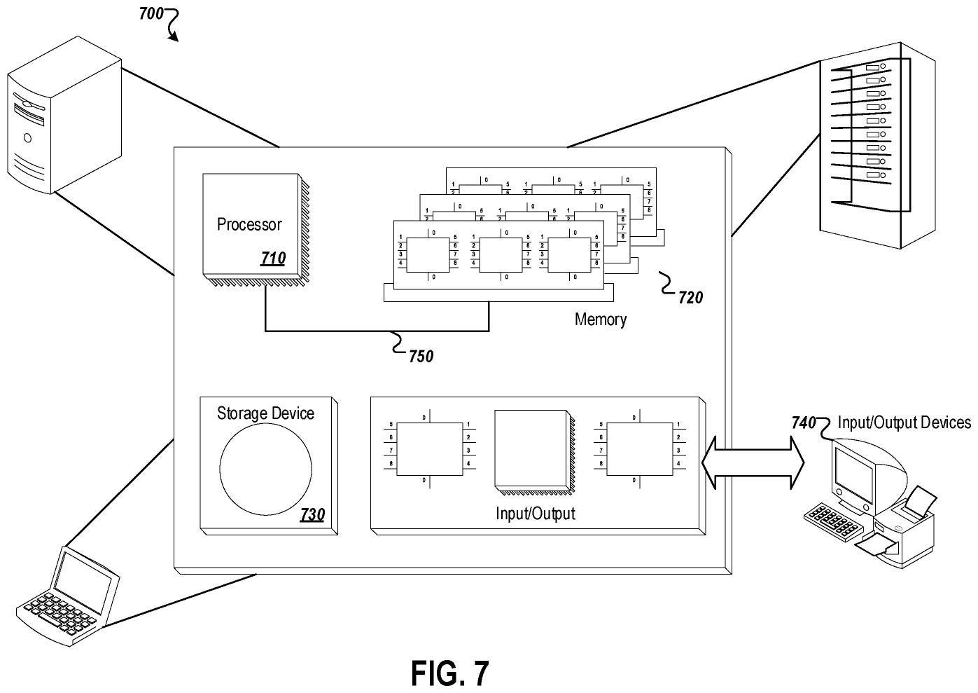

Generally, a computer will also include, or be operatively coupled to, one or more mass storage devices, and be configured to receive data from or transfer data to the mass storage devices. The mass storage devices can be, for example, magnetic, magneto-optical, or optical disks, or solid state drives. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few. FIG. 7 depicts an example computer 700 including a processor 710, memory 720, storage device 730, I/O devices 740, and a communications bus 750.

To provide for interaction with a user, the subject matter described in this specification can be implemented on one or more computers having, or configured to communicate with, a display device, e.g., a LCD (liquid crystal display) monitor, or a virtual-reality (VR) or augmented-reality (AR) display, for displaying information to the user, and an input device by which the user can provide input to the computer, e.g., a keyboard and a pointing device, e.g., a mouse, a trackball or touchpad. Other kinds of devices can be used to provide for interaction with a user as well; for example, feedback and responses provided to the user can be any form of sensory feedback, e.g., visual, auditory, speech or tactile; and input from the user can be received in any form, including acoustic, speech, or tactile input, including touch motion or gestures, or kinetic motion or gestures or orientation motion or gestures. In addition, a computer can interact with a user by sending documents to and receiving documents from a device that is used by the user; for example, by sending web pages to a web browser on a user's device in response to requests received from the web browser, or by interacting with an app running on a user device, e.g., a smartphone or electronic tablet. Also, a computer can interact with a user by sending text messages or other forms of message to a personal device, e.g., a smartphone that is running a messaging application, and receiving responsive messages from the user in return.

In this specification, the term “database” refers broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” refers broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

This specification uses the term “configured to” in connection with systems, apparatus, and computer program components. That a system of one or more computers is configured to perform particular operations or actions means that the system has on it software, firmware, hardware, or a combination of them that in operation cause the system to perform the operations or actions. That one or more computer programs is configured to perform particular operations or actions means that the one or more programs include instructions that, when executed by data processing apparatus, cause the apparatus to perform the operations or actions. That special-purpose logic circuitry is configured to perform particular operations or actions means that the circuitry has electronic logic that performs the operations or actions.

The subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some implementations, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of what is being claimed, which is defined by the claims themselves, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claim may be directed to a subcombination or variation of a subcombination.

Similarly, while operations are depicted in the drawings and recited in the claims in a particular order, this by itself should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method, comprising:
obtaining, by a system comprising one or more computers, patient profiles for a plurality of patients, wherein each patient profile corresponds to a particular patient and includes (i) values for an expanded set of predictive features about the particular patient and (ii) a target response classification that indicates whether the particular patient responded to a particular medical treatment according to one or more criteria;
training, by the system, a treatment prediction model using the patient profiles, including applying a machine-learning technique that causes the treatment prediction model to learn to predict, based on predictive features from a patient profile for a given patient, a likelihood that the given patient will respond to the particular medical treatment according to the one or more criteria;
identifying, by the system, a first subset of predictive features from the expanded set of predictive features that are most predictive of whether a given patient will respond to the particular medical treatment according to the one or more criteria;
configuring the treatment prediction model to generate predictions from values for the first subset of predictive features that are most predictive, without requiring values for a second subset of predictive features that are less predictive than features from the first subset; and
applying the treatment prediction model to generate a treatment response prediction for a new patient.

2. The method of claim 1, wherein the treatment prediction model is a random forest ensemble comprising a plurality of decision trees.

3. The method of claim 1, wherein the particular medical treatment is a treatment for alleviating chronic pain.

4. The method of claim 3, wherein the treatment for alleviating chronic pain is one of a multimodal treatment, a class of medication, a particular medication, a class of injection, a particular type of injection, an implanted medical device, a behavioral medicine treatment, an integrative medicine treatment, a rehabilitation therapy, or an orthotic or assistive device.

5. The method of claim 3, wherein the expanded set of predictive features include features indicating at least one of:
Charlson co-morbidity index,
post-traumatic stress disorder (PTSD),
pain experience duration,
opioid use or misuse,
neuropathic pain level,
Medicaid status,
socioeconomic factors in region of patient's residence,
medical diagnoses,
medication prescriptions,
medical procedure history,
body pain map,
patient age,
patient sex,
patient education level,
tobacco use,
alcohol use,
illicit drug use,
anxiety level,
depression level,
global mental health level,
global physical health level,
pain interference,
positive outlook level, or
sleep disturbance level.

6. The method of claim 1, wherein configuring the treatment prediction model to generate predictions from values for the first subset of predictive features that are most predictive comprises establishing a recommendation or a requirement that, after training, patient profiles for new patients cannot include missing values for the first subset of predictive features as a condition of using the treatment prediction model to generate treatment response predictions for the new patients.

7. The method of claim 1, wherein configuring the treatment prediction model to generate predictions from values for the first subset of predictive features that are most predictive comprises:
after initially training the treatment prediction model on the expanded set of predictive features, re-training the treatment prediction model only on the first subset of predictive features to exclusion of the second subset of predictive features.

8. The method of claim 7, wherein at least one of a size or a computational complexity of the treatment prediction model is reduced as a result of re-training the treatment prediction model.

9. The method of claim 1, further comprising:
identifying that the patient profile for a first particular patient is missing a value for a first feature in the expanded set of predictive features included in the patient profile; and
in response to identifying that the patient profile for the first particular patient is missing the value for the first feature, before using the patient profile for the first particular patient in training the treatment prediction model, imputing a value for the first feature.

10. The method of claim 9, wherein imputing the value for the first feature comprises:
if the first feature is a continuous variable, assigning an average value of the first feature from other patient profiles as the value for the first feature in the patient profile for the first particular patient; or
if the first feature is a discrete variable, assigning a null value as the value for the first feature in the patient profile for the first particular patient.

11. The method of claim 1, further comprising:
obtaining, by the system, updated patient profiles that include data related to additional patients, additional predictive features, or both, which were not in the patient profiles on which the treatment prediction model was previously trained;
identifying, by the system and based on the updated patient profiles, a third subset of predictive features that are most predictive of whether a given patient will respond to the particular medical treatment according to the one or more criteria; and
re-configuring the treatment prediction model to generate predictions from values for the third subset of predictive features that are most predictive.

12. The method of claim 11, wherein the third subset of predictive features includes at least one predictive feature that is not among the predictive features in the first subset; and
re-configuring the treatment prediction model comprises adding a recommendation or requirement that patient profiles for new patients cannot include missing values for the at least one predictive feature as a condition of using the treatment prediction model to generate treatment response predictions for the new patients.

13. The method of claim 11, wherein re-configuring the treatment prediction model comprises re-training the treatment prediction model only on the third subset of predictive features.

14. The method of claim 1, wherein the one or more criteria comprise achieving at least one of:
(i) a threshold improvement in average pain intensity within a predetermined time interval;
(ii) a threshold improvement in physical function within a predetermined time interval; or
(iii) a threshold improvement in patient's overall impression of change.

15. The method of claim 1, wherein training the treatment prediction model comprises achieving at least an area under receiver operating curve (AUROC) or selective area under receiver operating curve (SAUROC) score of 0.65.

16. A method, comprising:
obtaining, by a system comprising one or more computers, patient data that describes information about a patient and a medical condition of the patient;
generating, by the system and based on the patient data, a patient profile for the patient,
wherein the patient profile comprises values for a plurality of predictive features and the plurality of predictive features include one or more shared predictive features that are each processed by two or more treatment prediction models of a plurality of treatment prediction models,
wherein the plurality of treatment prediction models each corresponds to a different medical treatment modality of a plurality of medical treatment modalities;
generating treatment response predictions for the patient for each of the plurality of medical treatment modalities, including for each medical treatment modality:
processing, with the treatment prediction model that corresponds to the medical treatment modality, at least a subset of the plurality of predictive features from the patient profile to generate a treatment response prediction for the medical treatment modality,
wherein the two or more treatment prediction models each process the one or more shared predictive features to generate the treatment response predictions for the corresponding medical treatment modalities; and
outputting information about the treatment response predictions for the patient for one or more of the plurality of medical treatment modalities.

17. The method of claim 16, wherein each of the plurality of treatment prediction models is a separately trained random forest ensemble of decision trees.

18. The method of claim 16, wherein the plurality of medical treatment modalities are for alleviating chronic pain.

19. The method of claim 18, wherein the medical treatment modalities for alleviating chronic pain are selected from a group comprising a multimodal treatment, a class of medication, a particular medication, a class of injection, a particular type of injection, an implanted medical device, a behavioral medicine treatment, an integrative medicine treatment, a rehabilitation therapy, and an orthotic or assistive device.

20. The method of claim 18, wherein the plurality of predicted features include features indicating at least one of:
Charlson co-morbidity index,
post-traumatic stress disorder (PTSD),
pain experience duration,
opioid use or misuse,
neuropathic pain level,
Medicaid status,
socioeconomic factors in region of patient's residence,
medical diagnoses,
medication prescriptions,
medical procedure history,
body pain map,
patient age,
patient sex,
patient education level,
tobacco use,
alcohol use,
illicit drug use,
anxiety level,
depression level,
global mental health level,
global physical health level,
pain interference,
positive outlook level, or
sleep disturbance level.

21. The method of claim 16, wherein each of the plurality of treatment prediction models exhibits an area under receiver operating curve (AUROC) or selective area under receiver operating curve (SAUROC) score of at least 0.65.

22. The method of claim 16, wherein the plurality of predictive features include at least 2, 3, 4, 5, 6, 7, 8, 9, 10, 11, or 12 shared predictive features.

23. The method of claim 16, wherein the values for the one or more shared predictive features are each calculated once from the patient data and processed by each of the two or more treatment prediction models without need to re-calculate the values for the one or more shared predictive features for processing by different ones of the two or more treatment prediction models.

24. The method of claim 16, wherein a treatment response prediction indicates a likelihood of the patient achieving at least one of the following conditions within a predetermined period of time:
(i) a threshold improvement in average pain intensity,
(ii) a threshold improvement in physical function, or
(iii) a threshold improvement in patient's overall impression of change.

25. One or more computer-readable storage media encoded with instructions that, when executed by a system of one or more computers, cause the system to perform operations comprising:
obtaining, by the system, patient data that describes information about a patient and a medical condition of the patient;
generating, by the system and based on the patient data, a patient profile for the patient,
wherein the patient profile comprises values for a plurality of predictive features and the plurality of predictive features include one or more shared predictive features that are each processed by two or more treatment prediction models of a plurality of treatment prediction models,
wherein the plurality of treatment prediction models each corresponds to a different medical treatment modality of a plurality of medical treatment modalities;
generating treatment response predictions for the patient for each of the plurality of medical treatment modalities, including for each medical treatment modality:
processing, with the treatment prediction model that corresponds to the medical treatment modality, at least a subset of the plurality of predictive features from the patient profile to generate a treatment response prediction for the medical treatment modality,
wherein the two or more treatment prediction models each process the one or more shared predictive features to generate the treatment response predictions for the corresponding medical treatment modalities; and
outputting information about the treatment response predictions for the patient for one or more of the plurality of medical treatment modalities.
